(19) United States
(12) Patent Application Publication
(10) Pub. No.: US 2024/0425440 A1
(43) Pub. Date: Dec. 26, 2024

(54) METHOD FOR PRODUCING ACID GENERATOR

(71) Applicant: TOKYO OHKA KOGYO CO., LTD., Kawasaki-shi (JP)

(72) Inventors: Rin Odashima, Kawasaki-shi (JP); Hiroki Kato, Kawasaki-shi (JP)

(21) Appl. No.: 18/697,720

(22) PCT Filed: Oct. 20, 2022
(86) PCT No.: PCT/JP2022/039135

§ 371 Date: Apr. 1, 2024

(30) Foreign Application Priority Data
Oct. 22, 2021 (JP) 2021-173044

(51) Int. Cl. C07C 51/41; G03F 7/004

(52) U.S. Cl. CPC C07C 51/412; C07C 51/41; G03F 7/0045

(57) ABSTRACT

A method for producing an acid generator including reacting a carboxylic acid represented by Formula (d0-1) having a pKa of 0.50 or more with at least one of a nitrogen-containing base compound and an onium compound to obtain an intermediate represented by Formula (d0-p), and subjecting the intermediate (d0-p) to an ion exchange reaction with a compound represented by Formula (c0) to obtain a compound represented by Formula (d0). In the formulae, X⁰ represents a bromine atom or an iodine atom, R^{m} represents a hydroxy group, nb1 represents an integer in a range of 1 to 5, 1≤nb1+nb2≤5 is satisfied, Y^{d} represents a divalent linking group or a single bond, Mp^{m′+} represents an organic ammonium cation having a logP_{OW} of 4.8 or less or an onium cation having a logP_{OW} of 4.8 or less, X⁻ represents a counter anion, and M^{m+} represents an onium cation.

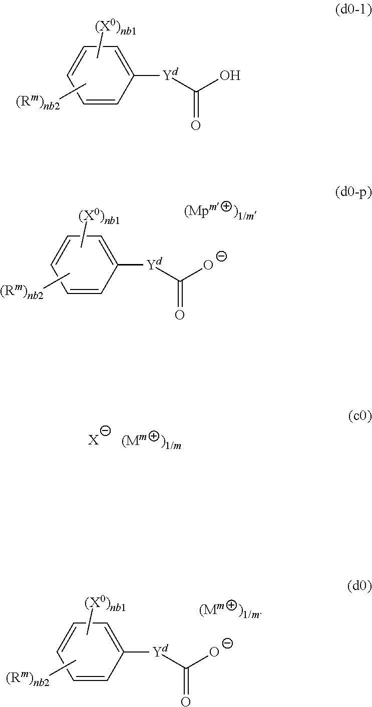

### TECHNICAL FIELD

The present invention relates to a method for producing an acid generator.

Priority is claimed on Japanese Patent Application No. 2021-173044, filed Oct. 22, 2021, the content of which is incorporated herein by reference.

### BACKGROUND ART

In recent years, in the production of semiconductor elements and liquid crystal display elements, advances in lithography technologies have led to rapid progress in the field of pattern miniaturization. Typically, these pattern miniaturization techniques involve shortening the wavelength (increasing the energy) of the light source for exposure.

Resist materials have been required to have lithography characteristics such as sensitivity to these light sources for exposure and resolution capable of reproducing a fine-sized pattern.

As a resist material that satisfies these requirements, a chemically amplified resist composition containing a base material component whose solubility in a developing solution is changed under action of an acid and an acid generator component (B) that generates an acid upon exposure to light is used in the related art.

In the resist pattern formation, the behavior of an acid generated from an acid generator component (B) upon exposure to light is considered as one factor that has a great influence on lithography characteristics. On the other hand, a chemically amplified resist composition having both an acid generator component (B) and an acid diffusion control agent component (D) that controls the diffusion of the acid generated from the acid generator component (B) upon exposure to light has been used.

As the acid diffusion control agent component (D) used in the chemically amplified resist composition, a nitrogen-containing organic compound such as an aliphatic amine or a cyclic amine, or a photodecomposable base that is decomposed upon exposure to light and loses the acid diffusion controllability (basicity) is known.

The photodecomposable base does not act as a quencher since the photodecomposable base loses the acid diffusion controllability (basicity) by decomposing the cation moiety and converting the anion moiety into an acid (that is, generating an acid) in the exposed portion of the resist film, but acts as a quencher in the unexposed portion of the resist film. Specifically, the photodecomposable base exhibits a quenching effect by causing an ion exchange reaction with the acid generated from the acid generator component (B) in the unexposed portion of the resist film. By blending such photodecomposable base, the diffusion of the acid generated from the acid generator component (B) from the exposed portion of the resist film to the unexposed portion is controlled, and the lithography characteristics are improved.

In the midst of further progress of lithography technology, expansion of application fields, and the like, in order to improve lithography characteristics, a wide variety of acid generator components (B) and acid diffusion control agent components (D) have been developed. In addition, there is a demand for a production method that enables these components to be obtained with a high yield.

For example, Patent Document 1 discloses a method for producing a second ammonium salt compound produced by reacting a first ammonium salt compound with a nitrogen-containing compound having a lone electron pair, in which the first ammonium salt compound contains a primary, secondary, or tertiary first ammonium cation, and a conjugate acid of the nitrogen-containing compound has an acid dissociation constant (pKa) greater than that of the first ammonium cation, and a method for producing a compound, including a step of performing salt exchange between the ammonium salt compound produced by the production method and a sulfonium cation or an iodonium cation having hydrophobicity higher than that of the conjugate acid of the nitrogen-containing compound. According to the method for producing the compound, it is said that an acid generator having less impurities can be obtained with a high yield.

### CITATION LIST

### Patent Document

### [Patent Document 1]

Japanese Unexamined Patent Application, First Publication No. 2014-15433

### SUMMARY OF INVENTION

### Technical Problem

In order to further improve the lithography characteristics, various studies have been conducted on components that can be blended in the resist composition. Among these, particularly, there is a demand for the molecular structure of an acid generator (an acid generator component (B), a photodecomposable base, or the like) that generates an acid upon exposure to light in the exposed portion of the resist film. Among these, for example, an onium salt-based acid generator having the relatively highly hydrophobic anion moiety has been developed.

However, for the onium salt-based acid generator having an anion moiety with a specific structure as described above, the yield is not sufficient in the production method of the related art as described in Patent Document 1, and a more efficient production method is required.

The present invention has been made in consideration of the above circumstances, and an object of the present invention is to provide a production method capable of producing an acid generator for a resist composition with a higher yield.

### Solution to Problem

In order to achieve the above-described object, the present invention adopts the following configurations.

That is, one aspect of the present invention is a method for producing an acid generator, the method including a step (I) of reacting a carboxylic acid represented by General Formula (d0-1) having an acid dissociation constant (pKa) of 0.50 or more with at least one selected from the group consisting of a nitrogen-containing base compound and an onium compound to obtain an intermediate represented by General Formula (d0-p) and a step (II) of subjecting the intermediate obtained in the step (I) to an ion exchange reaction with a compound represented by General Formula (c0) to obtain a compound represented by General Formula (d0).

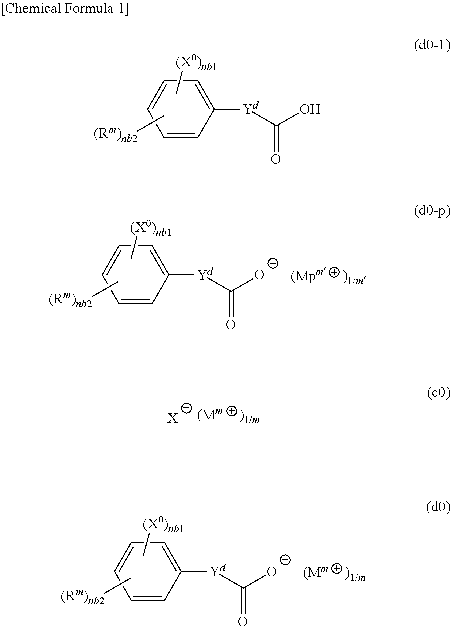

[In the formulae, X⁰ represents a bromine atom or an iodine atom. R^{m} represents a hydroxy group, an alkyl group, a fluorine atom, or a chlorine atom. nb1 represents an integer in a range of 1 to 5, and nb2 represents an integer in a range of 0 to 4, where 1≤nb1+nb2≤5 is satisfied. Y^{d} represents a divalent linking group or a single bond. Mp^{m′+} represents an organic ammonium cation having an octanol/water partition coefficient (logP_{OW}) of 4.8 or less or an onium cation having a logP_{OW} of 4.8 or less. m′ represents an integer of 1 or more. X⁻ represents a counter anion. M^{m+} represents an onium cation. m represents an integer of 1 or more.]

### Advantageous Effects of Invention

According to the method for producing an acid generator according to one aspect of the present invention, an acid generator for a resist composition can be produced with a higher yield.

### DESCRIPTION OF EMBODIMENTS

In the present specification and the scope of the present claims, the term “aliphatic” is a relative concept used with respect to “aromatic” and defines a group, a compound, or the like that has no aromaticity.

The term “alkyl group” includes a monovalent saturated hydrocarbon group that is linear, branched, or cyclic unless otherwise specified. The same applies to the alkyl group of an alkoxy group.

The term “alkylene group” includes a divalent saturated hydrocarbon group that is linear, branched, or cyclic unless otherwise specified.

Examples of the “halogen atom” include a fluorine atom, a chlorine atom, a bromine atom, and an iodine atom.

The expression “may have a substituent” includes both a case where a hydrogen atom (—H) is substituted with a monovalent group and a case where a methylene group (—CH₂—) is substituted with a divalent group.

In the present specification and the scope of the present claims, asymmetric carbons may be present and enantiomers or diastereomers may be present depending on the structures represented by the chemical formula. In that case, these isomers are representatively represented by one chemical formula. These isomers may be used alone or in the form of a mixture.

### (Method for Producing Acid Generator)

The method for producing an acid generator according to the present embodiment has the following step (I) and step (II).

Step (I): a step of reacting a carboxylic acid represented by General Formula (d0-1) having an acid dissociation constant (pKa) of 0.50 or more with at least one selected from the group consisting of a nitrogen-containing base compound and an onium compound to obtain an intermediate represented by General Formula (d0-p)

Step (II): a step of subjecting the intermediate obtained in the step (I) to an ion exchange reaction with a compound represented by General Formula (c0) to obtain a compound represented by General Formula (d0)

The acid generator produced by the production method according to the present embodiment is useful for a resist composition. The acid generator referred to herein is a compound that generates an acid upon exposure to light, and includes not only a compound that acts as an acid component but also a compound that acts relatively as a base component (an acid diffusion control agent).

Hereinafter, a method for producing an acid generator according to the present embodiment will be described in detail in order of the raw materials used in each step (at least one selected from the group consisting of a specific carboxylic acid, a nitrogen-containing base compound, and an onium compound, and a compound represented by General Formula (c0)), and the operations in the step (I) and the step (II).

### <<Specific Carboxylic Acid>>

In the step (I) in the production method according to the present embodiment, a carboxylic acid represented by General Formula (d0-1) and having an acid dissociation constant (pKa) of 0.50 or more (hereinafter, this carboxylic acid is also referred to as a specific carboxylic acid or the compound (d0-1)) is used.

The specific carboxylic acid (compound (d0-1)) may be used alone or in combination of two or more kinds thereof.

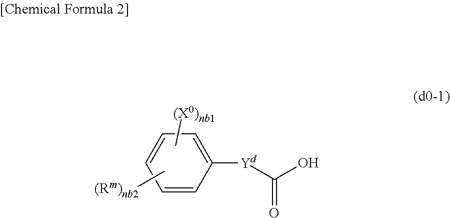

[In the formula, X⁰ represents a bromine atom or an iodine atom. R^{m} represents a hydroxy group, an alkyl group, a fluorine atom, or a chlorine atom. nb1 represents an integer of 1 to 5, and nb2 represents an integer of 0 to 4, where 1≤nb1+nb2≤5 is satisfied. Y^{d} represents a divalent linking group or a single bond.]

In General Formula (d0-1), X⁰ is a bromine atom or an iodine atom.

In General Formula (d0-1), R^{m} represents a hydroxy group, an alkyl group, a fluorine atom, or a chlorine atom. The alkyl group as R^{m} is preferably an alkyl group having 1 to 5 carbon atoms and more preferably a methyl group or an ethyl group. Among the above, R^{m} is preferably a hydroxy group.

In General Formula (d0-1), nb1 represents an integer of 1 to 5, nb2 represents an integer of 0 to 4, and 1≤nb1+nb2≤5 is satisfied.

It is preferable that nb1 represents an integer in a range of 1 to 3.

nb2 represents preferably an integer in a range of 0 to 3 and more preferably 0 or 1.

In General Formula (d0-1), Y^{d} represents a divalent linking group or a single bond.

Suitable examples of the divalent linking group as Y^{d} include a divalent linking group containing an oxygen atom. Examples of the divalent linking group having an oxygen atom include a non-hydrocarbon oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. A sulfonyl group (—SO₂—) may be further linked to the combination. In a case where Y^{d} is a divalent linking group containing an oxygen atom, Y^{d} may contain an atom other than the oxygen atom. Examples of the atom other than the oxygen atom include a carbon atom, a hydrogen atom, a sulfur atom, a nitrogen atom, and the like.

Among the above, Y^{d} is preferably a divalent linking group containing an oxygen atom or a single bond, and more preferably a single bond.

In the present invention, the “acid dissociation constant (pKa)” refers to the negative common logarithm (−logKa) of the equilibrium constant Ka, which is generally used as an index indicating the acid strength of a target substance.

The pKa of such specific carboxylic acid (compound (d0-1)) can be determined by measuring by a general method. In addition, as the pKa of such specific carboxylic acid (compound (d0-1)), a calculated value using a known software such as “ACD/Labs” (trade name, manufactured by Advanced Chemistry Development, Inc.) or “Chem3D” (trade name, manufactured by Hulinks Inc.) can also be used.

For the specific carboxylic acid (compound (d0-1)), the acid dissociation constant (pKa) is 0.50 or more, and the pKa is preferably 0.50 or more and 5.0 or less, more preferably 0.75 or more and 5.0 or less, and still more preferably 1.0 or more and 4.75 or less.

In a case where the pKa of the compound (d0-1) is equal to or more than the lower limit value of the above range, the final target product is easily produced with a higher yield, and in a case where the pKa is equal to or less than the upper limit value of the above range, the function as an acid generator for a resist composition is easily exhibited.

Specific examples of the suitable specific carboxylic acid (compound (d0-1)) are shown below.

The pKa shown together with the specific examples is a value calculated using the software “Chem3D ver15.1.0.144” (trade name, manufactured by Hulinks Inc.).

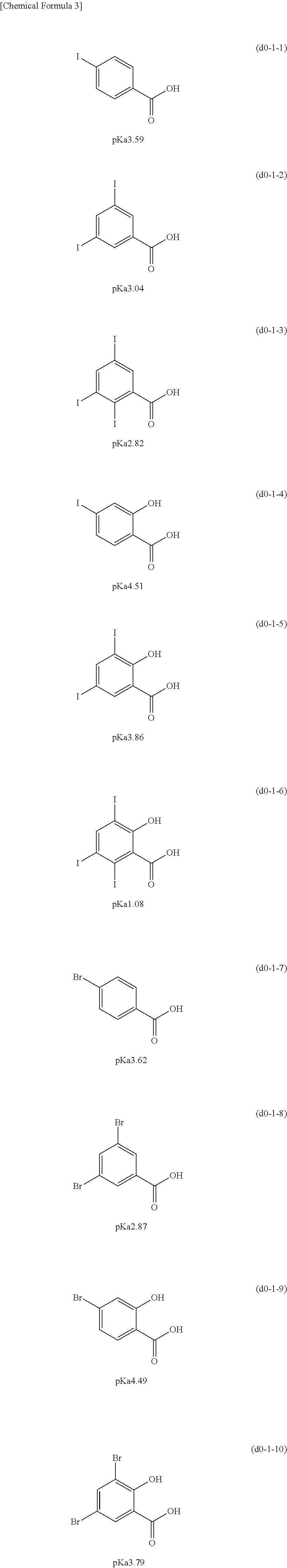

<<At Least One Selected from Group Consisting of Nitrogen-Containing Base Compound and Onium Compound>>

In the step (I) in the production method according to the present embodiment, at least one selected from the group consisting of a nitrogen-containing base compound and an onium compound (hereinafter, these are also collectively referred to as a compound (X0)) is used.

The compound (X0) may be used alone or in combination of two or more kinds thereof.

In the compound (X0), the onium compound does not include a compound corresponding the nitrogen-containing base compound.

In the present embodiment, examples of the compound (X0) include a compound represented by General Formula (X0).

(Mp^{m′⊕})_{1/m′}X′^{\textcircled{-}}(X0) [Chemical Formula 4]
[in the formula, Mp^{m′+} represents an organic ammonium cation having an octanol/water partition coefficient (logP_{OW}) of 4.8 or less or an onium cation having a logP_{OW} of 4.8 or less. m′ represents an integer of 1 or more. X′⁻ represents a counter anion.]

In the present invention, the “logP_{OW}” refers to a common logarithmic value of an octanol/water partition coefficient. “LogP_{OW}” is an effective parameter that can characterize the hydrophilicity/hydrophobicity of a wide range of compounds. Generally, the partition coefficient is determined by calculation regardless of an experiment, and in the present invention, the logP_{OW} shows a value calculated, for example, by CAChe Work System Pro Version 6.1. 12.33.

It means that the hydrophobicity increases in a case where the logP_{OW} increases on a positive side greater than 0, and the water solubility increases in a case where the absolute value increases on a negative side. The logP_{OW} has a negative correlation with the water solubility of an organic compound and is widely used as a parameter for estimating the hydrophilicity and hydrophobicity of an organic compound.

In General Formula (X0), Mp^{m′+} represents an organic ammonium cation having an octanol/water partition coefficient (logP_{OW}) of 4.8 or less or an onium cation having a logP_{OW} of 4.8 or less. m′ represents an integer of 1 or more.

For the organic ammonium cation in Mp^{m′+}, the logP_{OW} is 4.8 or less, the logP_{OW} is preferably −1.0 or more and 4.8 or less, the logP_{OW} is more preferably −1.0 or more and 3.0 or less, the logP_{OW} is still more preferably −1.0 or more and 2.0 or less, the logP_{OW} is particularly preferably −1.0 or more and 1.0 or less, and the logP_{OW} is most preferably −0.5 or more and 0 or less.

In a case where the logP_{OW} of the organic ammonium cation is equal to or less than the upper limit value of the above range, the final target product is easily produced with a higher yield, but on the other hand, in a case where the logP_{OW} of the organic ammonium cation is equal to or more than the lower limit value of the above range, the reaction efficiency of the step (I) is easily increased.

Examples of the organic ammonium cation as Mp^{m′+} include a cation represented by General Formula (ca-p1) and a cation represented by General Formula (ca-p2).

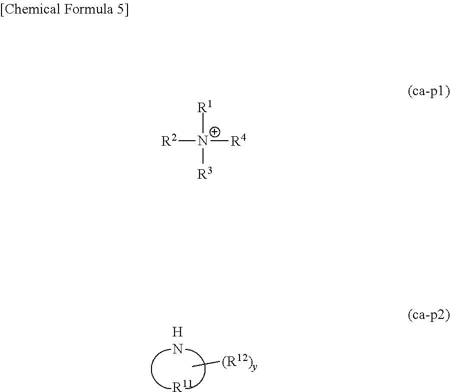

[In the formulae, R¹ to R⁴ each independently represent a hydrocarbon group which may have a substituent or a hydrogen atom. Provided that at least one of R¹ to R⁴ represents a hydrocarbon group which may have a substituent. Alternatively, at least two of R¹ to R⁴ may be bonded to each other to form an alicyclic structure together with the nitrogen atom in the formula. R¹¹ represents a group that forms an aromatic ring together with the nitrogen atom to which R^{m} is bonded. R¹² represents an alkyl group or a halogen atom. y is an integer in a range of 0 to 5.]

In General Formula (ca-p1), the hydrocarbon groups as R¹ to R⁴ are each independently preferably a hydrocarbon group having 1 to 15 carbon atoms and more preferably a hydrocarbon group having 1 to 10 carbon atoms. In addition, the total number of carbon atoms in the hydrocarbon group as R¹ to R⁴ is preferably in a range of 1 to 20, more preferably in a range of 3 to 18, and still more preferably in a range of 4 to 15.

Examples of the hydrocarbon group as R¹ to R⁴ include a linear or branched alkyl group and a cyclic hydrocarbon group.

The linear or branched alkyl group is preferably a linear or branched alkyl group having 1 to 10 carbon atoms and more preferably a linear or branched alkyl group having 1 to 5 carbon atoms.

The cyclic hydrocarbon group may be an alicyclic hydrocarbon group or an aromatic hydrocarbon group.

The alicyclic hydrocarbon group is preferably a group obtained by removing one hydrogen atom from a monocycloalkane. The monocycloalkane has preferably 3 to 6 carbon atoms, and specific examples thereof include cyclopentane and cyclohexane. The aromatic hydrocarbon group is preferably a phenyl group or a benzyl group.

Examples of the substituent which may be included in the hydrocarbon group as R¹ to R⁴ include an alkoxy group, a hydroxyl group, an oxo group (═O), and an amino group.

In General Formula (ca-p2), R¹¹ represents a group that forms an aromatic ring together with the nitrogen atom to which the R^{m} is bonded. The aromatic ring is preferably a 4- to 7-membered ring, more preferably a 4- to 6-membered ring, and still more preferably a 6-membered ring.

In General Formula (ca-p2), examples of the alkyl group as R¹² include the same one as the linear or branched alkyl group as R¹ to R⁴ described above.

In General Formula (ca-p2), y represents an integer in a range of 0 to 5, preferably 0 or 1, and more preferably 0.

Hereinafter, specific examples of the organic ammonium cation in Mp^{m′+} as the cation moiety of the compound (X0) will be shown. Together, the logP_{OW} value calculated by CAChe Work System Pro Version 6.1.12.33 is shown for each organic ammonium cation.

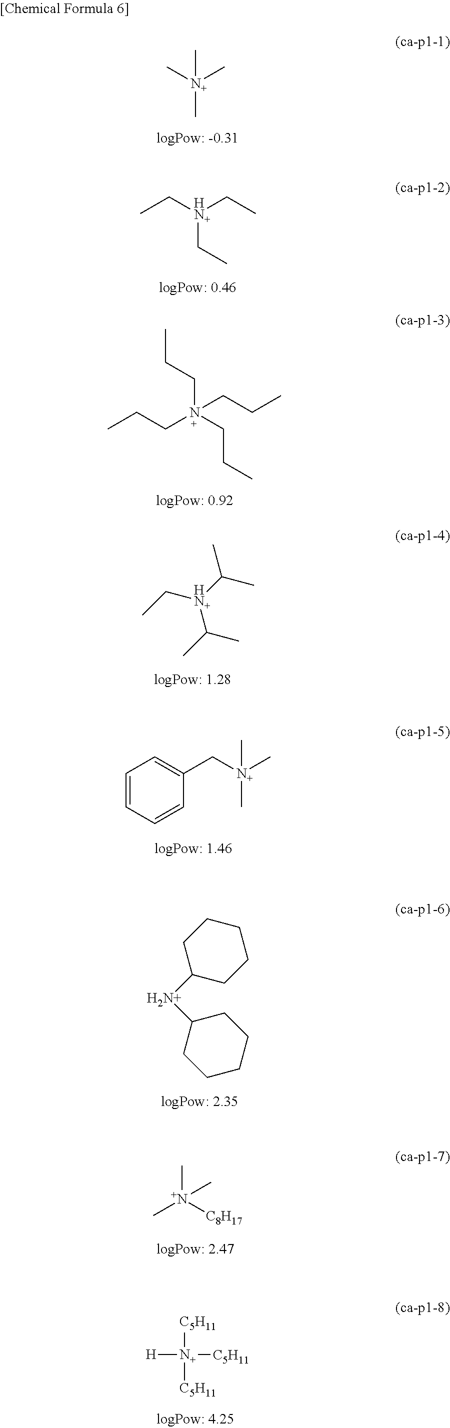

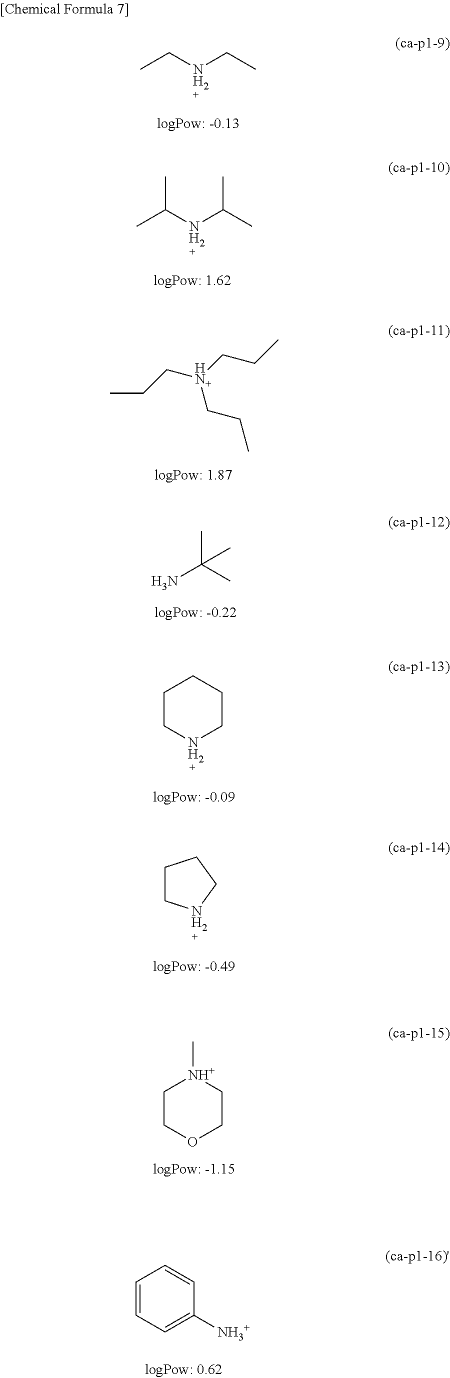

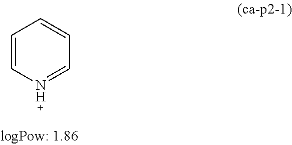

For the onium cation in Mp^{m′+}, the logP_{OW} is 4.8 or less, the logP_{OW} is preferably −1.0 or more and 4.8 or less, the logP_{OW} is more preferably −1.0 or more and 3.0 or less, the logP_{OW} is still more preferably −1.0 or more and 2.0 or less, the logP_{OW} is particularly preferably −1.0 or more and 1.5 or less, and the logP_{OW} is most preferably −0.5 or more and 0.5 or less.

In a case where the log Pow of the organic ammonium cation is equal to or less than the upper limit value of the above range, the final target product is easily produced with a higher yield, and in a case where the log Pow of the organic ammonium cation is equal to or more than the lower limit value of the above range, the function as an acid generator for a resist composition (particularly an acid diffusion control agent) is easily exhibited.

Examples of the onium cation as Mp^{m′+} include a cation represented by General Formula (ca-p3) and a cation represented by General Formula (ca-p4).

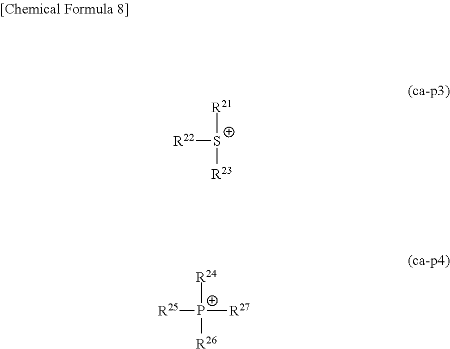

[In the formula, R²¹ to R²³ each independently represent a hydrocarbon group which may have a substituent. R²⁴ to R²⁷ each independently represent a hydrocarbon group which may have a substituent.]

In General Formula (ca-p3), the description of the hydrocarbon group as R²¹ to R²³ and the description of the substituent which may be contained in the hydrocarbon group are the same as the description of the hydrocarbon group as R¹ to R⁴ and the description of the substituent which may be contained in the hydrocarbon group in General Formula (ca-p1) described above.

The hydrocarbon group as each of R²¹ to R²³ is preferably a linear or branched alkyl group, more preferably a linear or branched alkyl group having 1 to 10 carbon atoms, and still more preferably a linear or branched alkyl group having 1 to 5 carbon atoms.

In General Formula (ca-p4), the description of the hydrocarbon group as R²⁴ to R²⁷ and the description of the substituent which may be contained in the hydrocarbon group are the same as the description of the hydrocarbon group as R¹ to R⁴ and the description of the substituent which may be contained in the hydrocarbon group in General Formula (ca-p1) described above.

The hydrocarbon group as each of R²⁴ to R²⁷ is preferably a linear or branched alkyl group, more preferably a linear or branched alkyl group having 1 to 10 carbon atoms, and still more preferably a linear or branched alkyl group having 1 to 5 carbon atoms.

Hereinafter, specific examples of the onium cation Mp^{m′+}, as the cation moiety of the compound (X0) will be shown. Together, the logP_{OW} value calculated by CAChe Work System Pro Version 6.1.12.33 is shown for each onium cation.

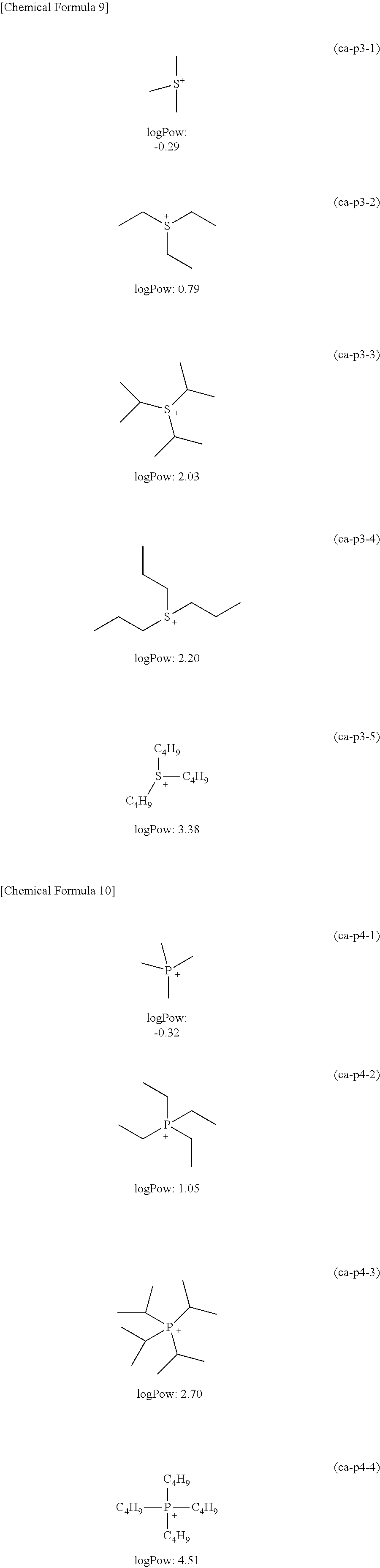

In General Formula (X0), examples of the counter anion as X′⁻ include a hydroxide ion (OH⁻).

The compound (X0) according to the present embodiment is at least one selected from the group consisting of a nitrogen-containing base compound and an onium compound, and among the above, at least one selected from the group consisting of a hydroxide of a cation represented by General Formula (ca-p1) and a hydroxide ion, a hydroxide of a cation represented by General Formula (ca-p2) and a hydroxide ion, a hydroxide of a cation represented by General Formula (ca-p3) and a hydroxide ion, and a hydroxide of a cation represented by General Formula (ca-p4) and a hydroxide ion is preferable, and at least one selected from the group consisting of a hydroxide of a cation represented by General Formula (ca-p1) and a hydroxide ion, a hydroxide of a cation represented by General Formula (ca-p3) and a hydroxide ion, and a hydroxide of a cation represented by General Formula (ca-p4) and a hydroxide ion is more preferable.

<<Compound Represented by General Formula (c0)>>

In the step (II) in the production method according to the present embodiment, a compound represented by General Formula (c0) (compound (c0)) is used as a compound for an ion exchange reaction.

The compound (c0) may be used alone or in combination of two or more kinds thereof.

[Chemical Formula 11]

x^{\textcircled{-}}(M^{m⊕})_{1/m} (c0)

[In the formula, X⁻ represents a counter anion. M^{m+} represents an onium cation. m represents an integer of 1 or more.]

In General Formula (c0), X⁻ represents a counter anion. Examples of X⁻ include ions that can be an acid having a lower acidity than the intermediate represented by General Formula (d0-p), and specific examples thereof include a halogen ion such as a bromine ion or a chloride ion, BF₄⁻, AsF_{b}⁻, SbF₆⁻, PF6⁻, ClO₄⁻, and the like. Among these, X⁻ is preferably a halogen ion and more preferably a chlorine ion.

In General Formula (c0), M^{m+} is an onium cation. m represents an integer of 1 or more. Examples of the onium cation in M^{m+} include a sulfonium cation and an iodonium cation.

Preferred examples of the cation moiety ((M^{m+})_{1/m}) include organic cations each represented by General Formulae (ca-1) to (ca-5).

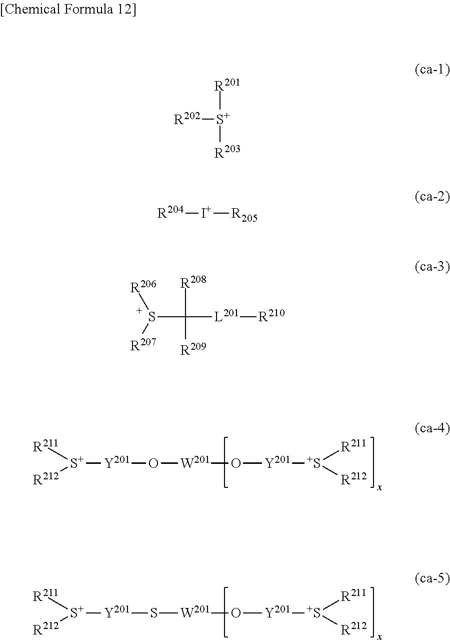

[In the formulae, R²⁰¹ to R²⁰⁷, R²¹¹, and R²¹² each independently represent an aryl group which may have a substituent, an alkyl group which may have a substituent, or an alkenyl group which may have a substituent. R²⁰¹ to R²⁰³, R²⁰⁶ and R²⁰⁷, and R²¹¹ and R²¹² may be bonded to each other to form a ring with the sulfur atom in the formulae. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a SO₂—-containing cyclic group which may have a substituent. L²⁰¹ represents —C(═O)— or —C(═O)—O—. Y²⁰¹′s each independently represent an arylene group, an alkylene group, or an alkenylene group. x represents 1 or 2. W²⁰¹ represents an (x+1)-valent linking group.]

In General Formulae (ca-1) to (ca-5), examples of the aryl group as R²⁰¹ to R²⁰⁷, R²¹¹, and R²¹² include an unsubstituted aryl group having 6 to 20 carbon atoms. Among these, a phenyl group or a naphthyl group is preferable.

The alkyl group as R²⁰¹ to R²⁰⁷, R²¹¹, and R²¹² is a chain-like or cyclic alkyl group, and the number of carbon atoms thereof is preferably in a range of 1 to 30.

It is preferable that the alkenyl group as R²⁰¹ to R²⁰⁷, R²¹¹, and R²¹² has 2 to 10 carbon atoms.

Examples of the substituent that R²⁰¹ to R²⁰⁷ and R²¹⁰ to R²¹² may have include an alkyl group, a halogen atom, a halogenated alkyl group, a carbonyl group, a cyano group, an amino group, an aryl group, and groups each represented by General Formulae (ca-r-1) to (ca-r-7).

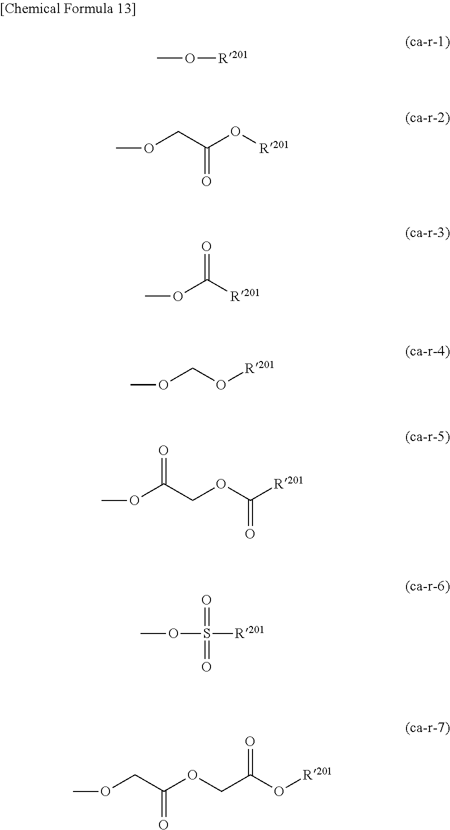

[In the formulae, R′²⁰¹′s each independently represent a hydrogen atom, a cyclic group which may have a substituent, a chain-like alkyl group which may have a substituent, or a chain-like alkenyl group which may have a substituent.]

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group with no aromaticity. In addition, the aliphatic hydrocarbon group may be saturated or unsaturated. Typically, it is preferable that the aliphatic hydrocarbon group is saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Provided that the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with hetero atoms. Examples of the hetero atom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. The monocyclic alicyclic hydrocarbon group is preferably a group obtained by removing one or more hydrogen atoms from a monocycloalkane. The monocycloalkane has preferably 3 to 6 carbon atoms, and specific examples thereof include cyclopentane and cyclohexane. The polycyclic alicyclic hydrocarbon group is preferably a group obtained by removing one or more hydrogen atoms from a polycycloalkane, and the polycycloalkane preferably has 7 to 30 carbon atoms. Among these, a polycycloalkane having a crosslinked ring polycyclic skeleton such as adamantane, norbornane, isobornane, tricyclodecane, or tetracyclododecane; and a polycycloalkane having a condensed ring-based polycyclic skeleton such as a cyclic group having a steroid skeleton are preferable as the polycycloalkane.

Among these, the cyclic aliphatic hydrocarbon group as R′²⁰¹ is preferably a group obtained by removing one or more hydrogen atoms from a monocycloalkane or a polycycloalkane, more preferably a group obtained by removing one hydrogen atom from a polycycloalkane, particularly preferably an adamantyl group or a norbornyl group, and most preferably an adamantyl group.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

In addition, the cyclic hydrocarbon group as R′²⁰¹ may have a hetero atom such as a heterocyclic ring. Specific examples thereof include lactone-containing cyclic groups each represented by General Formulae (a2-r-1) to (a2-r-7), —SO₂—-containing cyclic groups each represented by General Formulae (a5-r-1) to (a5-r-4), and heterocyclic groups each represented by Chemical Formulae (r-hr-1) to (r-hr-16). In the chemical formulae, * represents a bonding site.

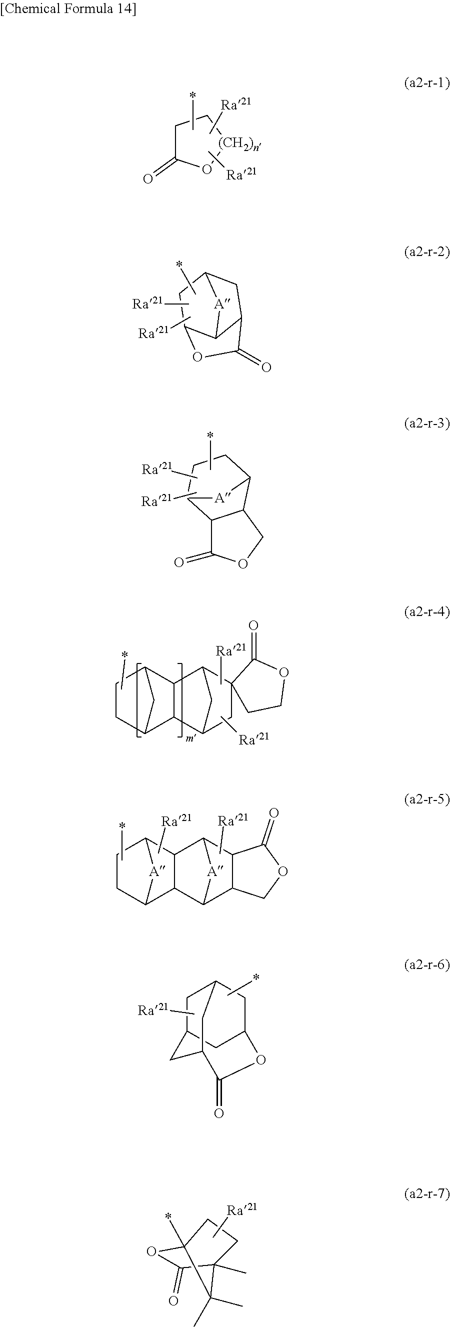

[In the formulae, Ra′²¹′s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, a lactone-containing cyclic group, a carbonate-containing cyclic group, or a —SO₂—-containing cyclic group, A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ represents 0 or 1. * represents a bonding site].

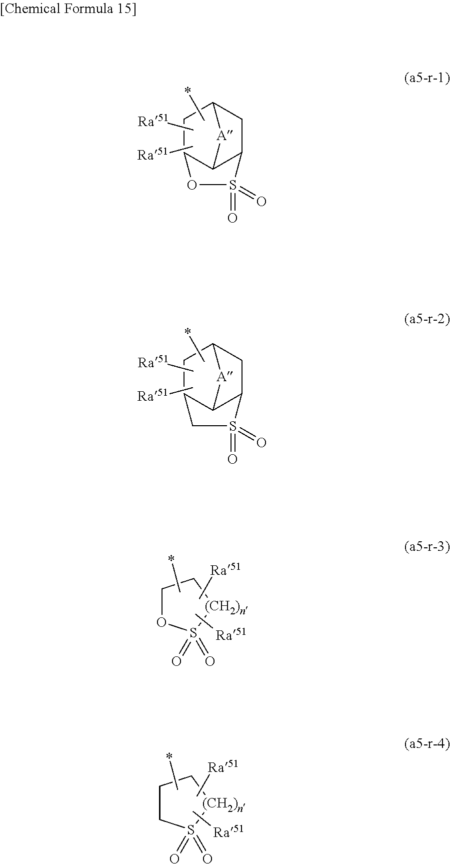

[In the formulae, Ra′⁵¹′s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R^{m} represents a hydrogen atom, an alkyl group, a lactone-containing cyclic group, a carbonate-containing cyclic group, or a —SO₂—-containing cyclic group, A″ represents an alkylene group having 1 to 5 carbon atoms which may have an oxygen atom or a sulfur atom, an oxygen atom, or a sulfur atom, and n′ represents an integer of 0 to 2. * represents a bonding site].

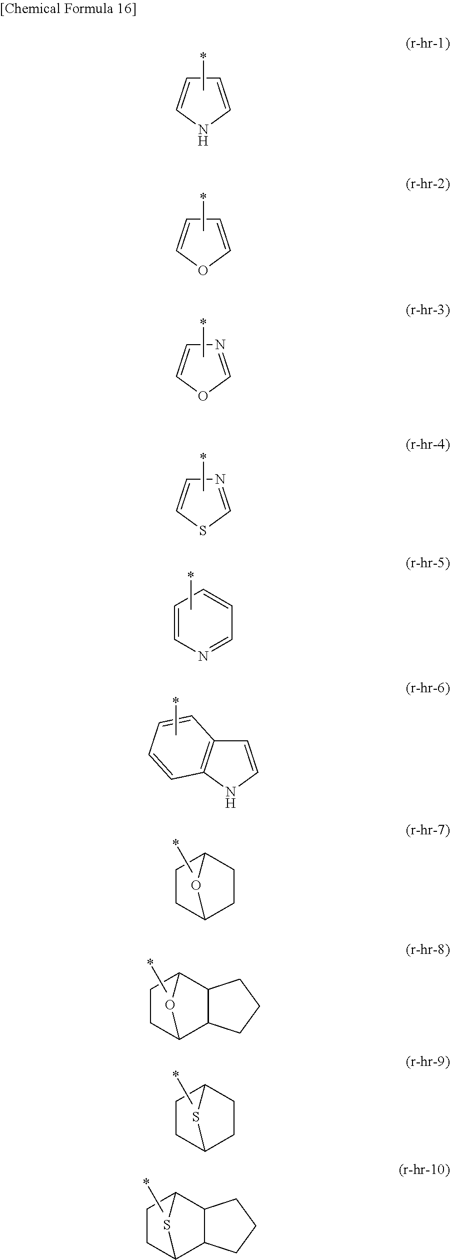

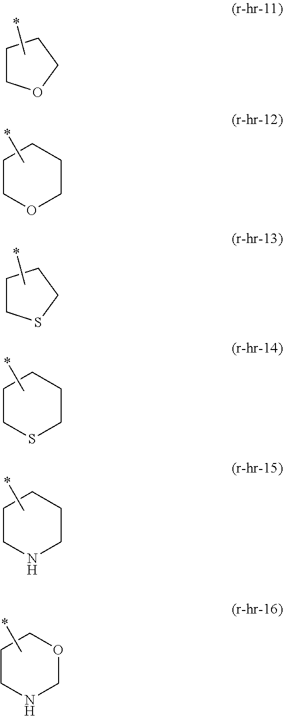

In General Formulae (a2-r-1) to (a2-r-7), it is preferable that the alkyl group as Ra′²¹ is an alkyl group having 1 to 6 carbon atoms. The alkyl group is preferably a linear alkyl group or a branched alkyl group. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ is an alkoxy group having 1 to 6 carbon atoms. It is preferable that the alkoxy group is linear or branched. Specific examples of the alkoxy groups include a group formed by linking the above-described alkyl group exemplified as the alkyl group represented by Ra′²¹ to an oxygen atom (—O—).

The halogen atom as Ra′²¹ is preferably a fluorine atom.

Examples of the halogenated alkyl group as Ra′²¹ include groups in which some or all hydrogen atoms in the alkyl group as Ra′^{2L} have been substituted with the halogen atoms. The halogenated alkyl group is preferably a fluorinated alkyl group and particularly preferably a perfluoroalkyl group.

In —COOR″ and —OC(═O)R″ as Ra′²¹, each R″ represents a hydrogen atom, an alkyl group, a lactone-containing cyclic group, a carbonate-containing cyclic group, or a —SO₂—-containing cyclic group.

The alkyl group as R″ may be linear, branched, or cyclic and has preferably 1 to 15 carbon atoms. In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable. In a case where R″ represents a cyclic alkyl group, the cyclic alkyl group preferably has 3 to 15 carbon atoms, more preferably has 4 to 12 carbon atoms, and most preferably has 5 to 10 carbon atoms. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane, or tetracyclododecane.

The “lactone-containing cyclic group” as R^{m} indicates a cyclic group that contains a ring (lactone ring) containing a —O—C(═O)— in the ring skeleton. The lactone ring is counted as the first ring, and in a case where the group contains only the lactone ring, the group is referred to as a monocyclic group, or in a case where the group further has other ring structures, the group is referred to as a polycyclic group regardless of the structures. The lactone-containing cyclic group may be a monocyclic group or a polycyclic group. The lactone-containing cyclic group in R″ is not particularly limited, and any constitutional unit can be used. Specific examples thereof include the same groups as those represented by General Formulae (a2-r-1) to (a2-r-7).

The “carbonate-containing cyclic group” as R^{m} indicates a cyclic group having a ring (a carbonate ring) containing —O—C(═O)—O— in the ring skeleton thereof. In a case where the carbonate ring is counted as the first ring and the group has only the carbonate ring, the group is referred to as a monocyclic group. Further, in a case where the group has other ring structures, the group is referred to as a polycyclic group regardless of the structures. The carbonate-containing cyclic group may be a monocyclic group or a polycyclic group.

The “—SO₂—-containing cyclic group” as R″ denotes a cyclic group having a ring containing —SO₂— in the ring skeleton thereof. Specifically, the —SO₂—-containing cyclic group is a cyclic group in which the sulfur atom (S) in —SO₂— forms a part of the ring skeleton of the cyclic group. In a case where the ring containing —SO₂— in the ring skeleton thereof is counted as the first ring and the group has only the ring, the group is referred to as a monocyclic group. In a case where the group further has other ring structures, the group is referred to as a polycyclic group regardless of the structures. The —SO₂—-containing cyclic group may be a monocyclic group or a polycyclic group.

The —SO₂—-containing cyclic group is particularly preferably a cyclic group containing —O—SO₂— in the ring skeleton thereof, that is, a cyclic group containing a sultone ring in which —O—S— in —O—SO₂— forms a part of the ring skeleton thereof. More specific examples of the —SO₂—-containing cyclic group include the same groups as those represented by General Formulae (a5-r-1) to (a5-r-4).

The hydroxyalkyl group as Ra′²¹ preferably has 1 to 6 carbon atoms, and specific examples thereof include a group in which at least one hydrogen atom in the alkyl group as Ra′²¹ has been substituted with a hydroxyl group.

Among the examples, it is preferable that Ra′²¹′s each independently represent a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. In a case where the alkylene group has an oxygen atom or a sulfur atom, specific examples thereof include groups in which —O— or —S— is interposed in the terminal of the alkylene group or between the carbon atoms of the alkylene group. Further, examples thereof include —O—CH₂—, —CH₂—O—CH₂—, —S—CH₂—, and —CH₂—S—CH₂—. A″ represents preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below. In the chemical formula showing a specific example, * represents a bonding site.

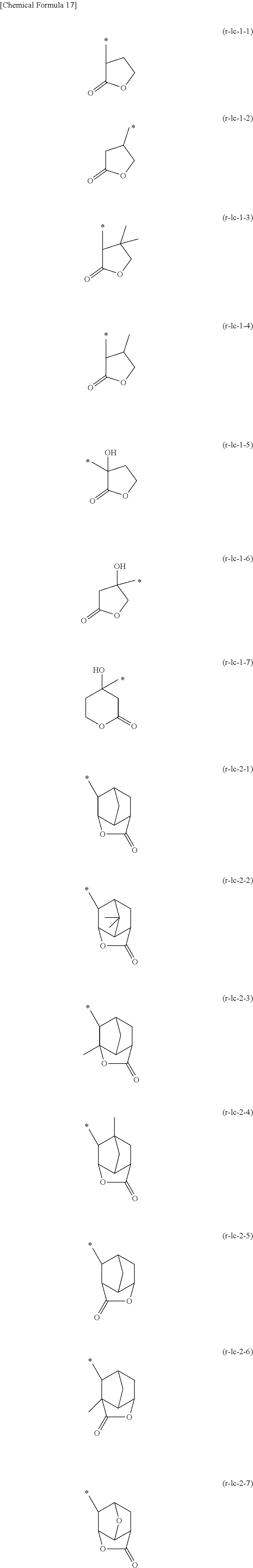

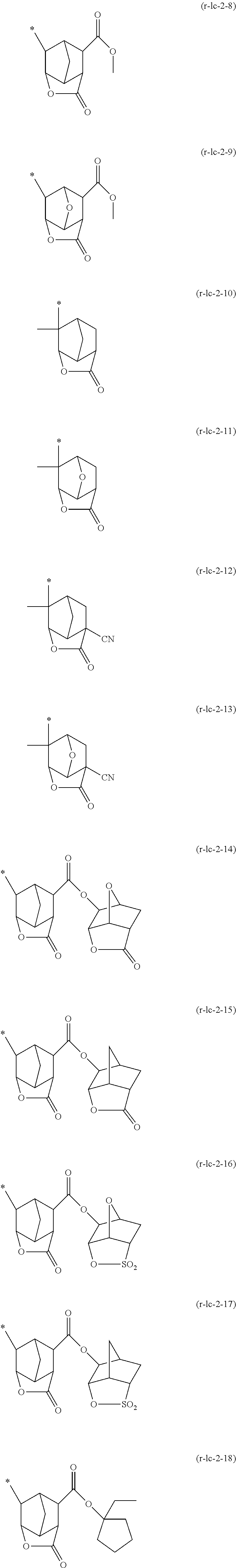

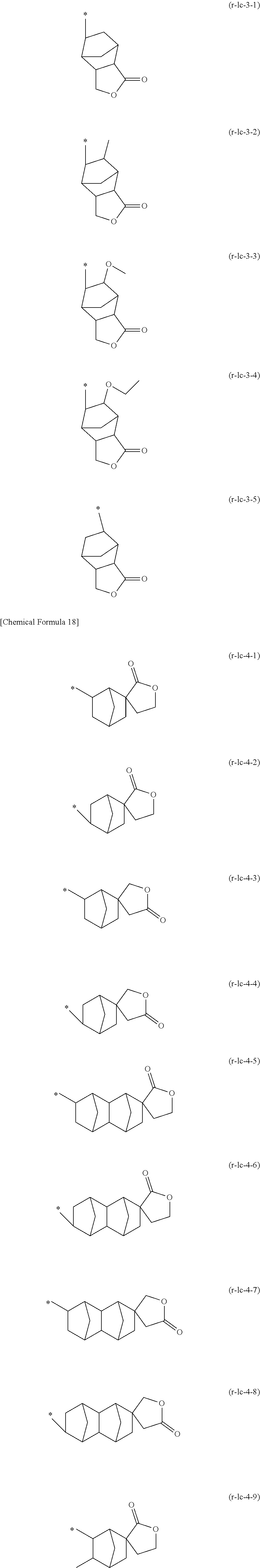

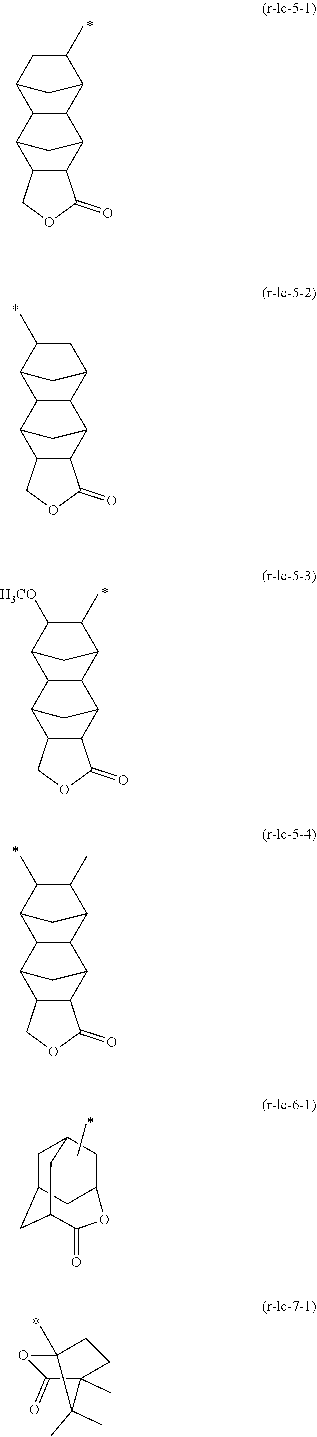

In General Formulae (a5-r-1) and (a5-r-2), A″ has the same definition as that for A″ in General Formulae (a2-r-2), (a2-r-3) and (a2-r-5).

Examples of the alkyl group, the alkoxy group, the halogen atom, the halogenated alkyl group, —COOR″, —OC(═O)R″, and the hydroxyalkyl group as Ra′⁵¹ include the same groups as those for Ra′²¹ in General Formulae (a2-r-1) to (a2-r-7).

Specific examples of the groups each represented by General Formulae (a5-r-1) to (a5-r-4) are shown below. In the formulae shown below, “Ac” represents an acetyl group. * represents a bonding site.

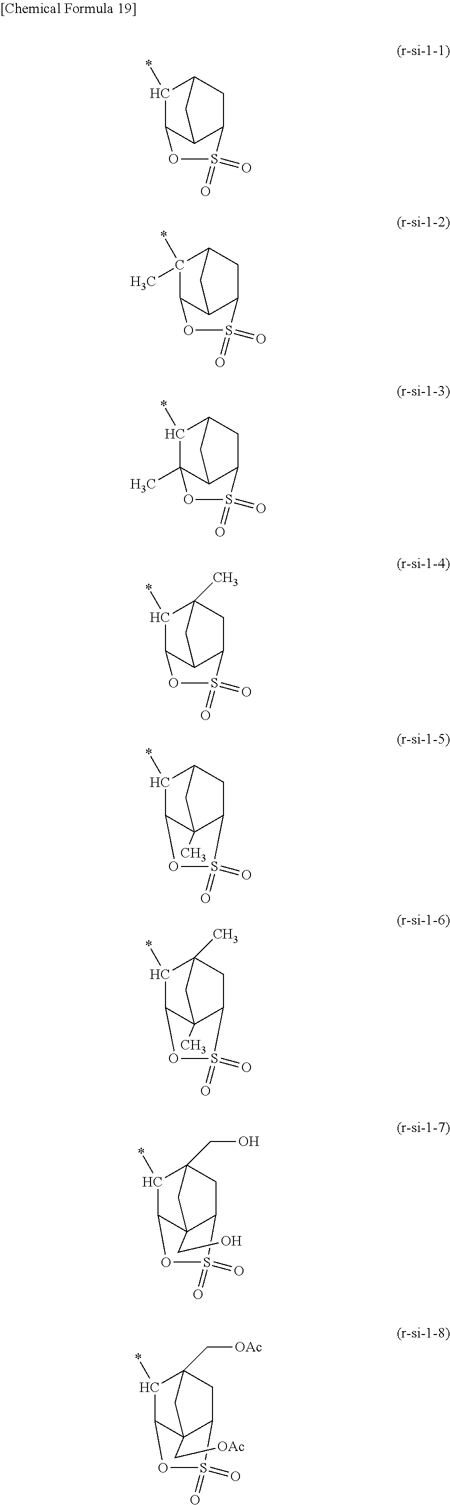

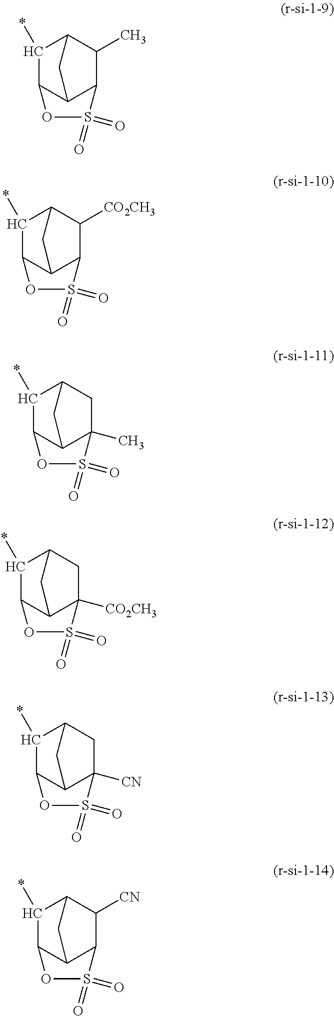

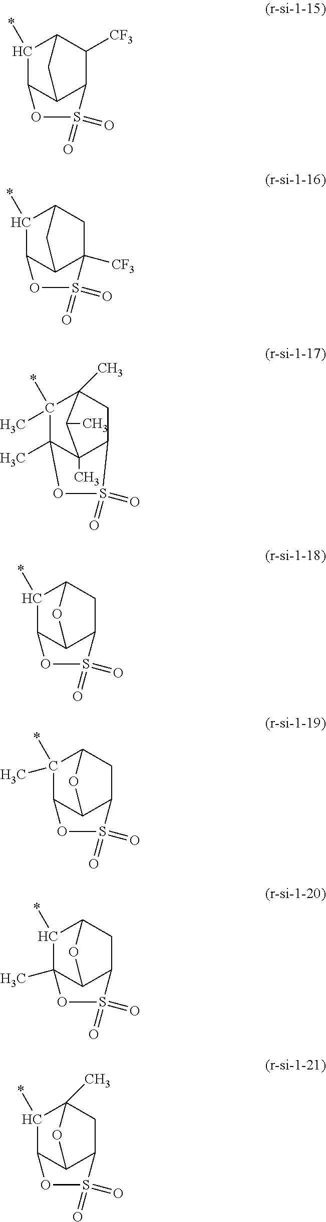

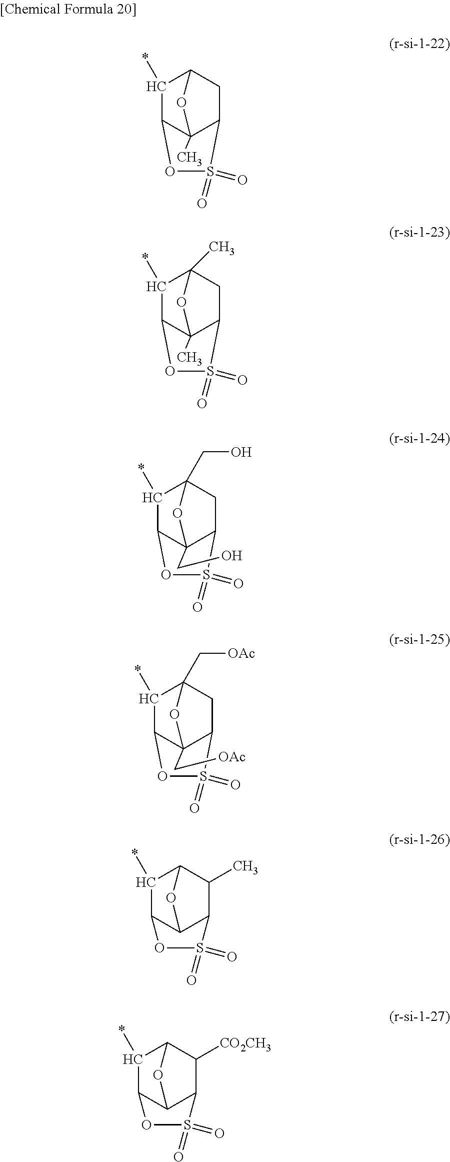

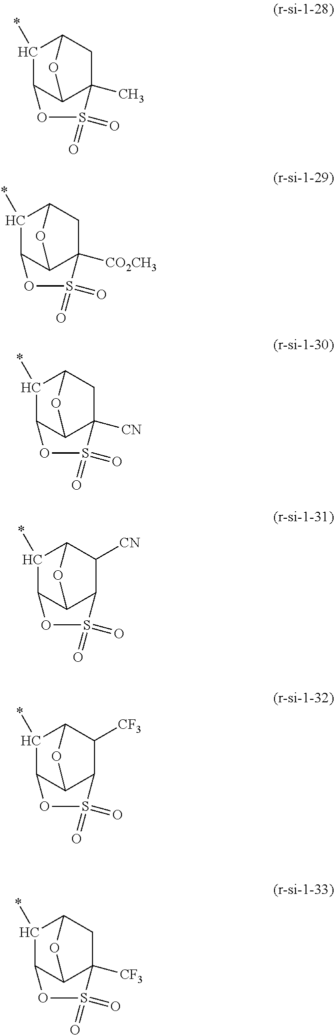

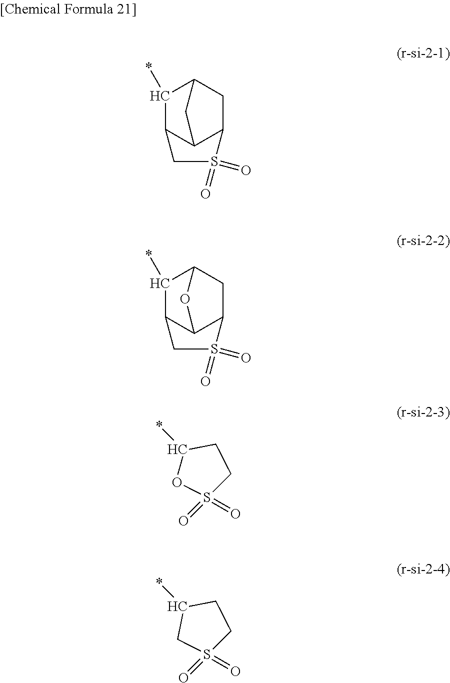

Examples of the substituent for the cyclic group as R′²⁰¹ include an alkyl group, an alkoxy group, a halogen atom, a halogenated alkyl group, a hydroxyl group, a carbonyl group, and a nitro group.

The alkyl group as the substituent is preferably an alkyl group having 1 to 5 carbon atoms, and a methyl group, an ethyl group, a propyl group, an n-butyl group, or a tert-butyl group is most preferable.

The alkoxy group as the substituent preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

As the halogen atom as a substituent, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

Chain-like alkyl group which may have substituent:

The chain-like alkyl group as R²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-like alkenyl group which may have substituent:

The chain-like alkenyl group as R′²⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butenyl group. Examples of the branched alkenyl group include a 1-methylvinyl group, a 2-methylvinyl group, a 1-methylpropenyl group, and a 2-methylpropenyl group.

Among the above, the chain-like alkenyl group is preferably a linear alkenyl group, more preferably a vinyl group or a propenyl group, and particularly preferably a vinyl group.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

The cyclic group which may have a substituent, the chain-like alkyl group which may have a substituent, or the chain-like alkenyl group which may have a substituent, as R′²⁰¹, in addition to those described above, may be an acid dissociable group represented by General Formula (a1-r-2) as a cyclic group which may have a substituent or a chain-like alkyl group which may have a substituent.

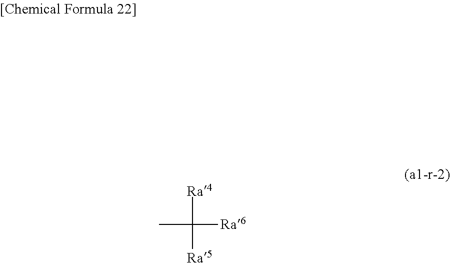

[In the formula, Ra′⁴ to Ra′⁶ each represent a hydrocarbon group, and Ra′⁵ and Ra′⁶ may be bonded to each other to form a ring.]

Examples of the hydrocarbon group as Ra′⁴ to Ra′⁶ include a linear or branched alkyl group, a chain-like or cyclic alkenyl group, and a cyclic hydrocarbon group.

Among the examples, R′²⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂—-containing cyclic group represented by any of General Formulae (a5-r-1) to (a5-r-4).

In General Formulae (ca-1) to (ca-5), in a case where R²⁰¹ to R²⁰³, R²⁰⁶ and R²⁰⁷, and R²¹¹ and R²¹² are bonded to each other to form a ring with a sulfur atom in the formula, these groups may be bonded to each other via a hetero atom such as a sulfur atom, an oxygen atom, or a nitrogen atom, or a functional group such as a carbonyl group, —SO—, —SO₂—, —SO₃—, —COO—, —CONH— or —N(RN)— (here, RN represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a henoxathiine ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a SO₂—-containing cyclic group which may have a substituent.

Examples of the aryl group as R²¹⁰ include an unsubstituted aryl group having 6 to 20 carbon atoms. Among these, a phenyl group or a naphthyl group is preferable.

As the alkyl group as R²¹⁰, a chain-like or cyclic alkyl group having 1 to 30 carbon atoms is preferable.

It is preferable that the alkenyl group as R²¹⁰ has 2 to 10 carbon atoms.

As the SO₂—-containing cyclic group as R²¹⁰ which may have a substituent, “—SO₂—-containing polycyclic group” is preferable, and a group represented by General Formula (a5-r-1) is more preferable.

Y²⁰¹′s each independently represent an arylene group, an alkylene group, or an alkenylene group.

Examples of the arylene group as Y²⁰¹ include an unsubstituted arylene group having 6 to 20 carbon atoms.

Examples of the alkylene group and the alkenylene group as Y²⁰¹ include an unsubstituted alkylene group and an unsubstituted alkenylene group, which have 6 to 20 carbon atoms.

In Formula (ca-4), x represents 1 or 2.

W²⁰¹ represents an (x+1)-valent linking group, that is, a divalent or trivalent linking group.

Examples of the divalent linking group as W²⁰¹ include a divalent hydrocarbon group which may have a substituent. The divalent linking group as W²⁰¹ may be any of linear, branched, or cyclic and is preferably cyclic. Among these, a group in which two carbonyl groups are combined with both ends of the arylene group is preferable. Examples of the arylene group include a phenylene group and a naphthylene group. Among these, a phenylene group is particularly preferable.

Examples of the trivalent linking group as W²⁰¹ include a group in which one hydrogen atom has been removed from the above-described divalent linking group as W²⁰¹ and a group obtained by bonding the divalent linking group to another divalent linking group described above. As the trivalent linking group as W²⁰¹, a group obtained by bonding two carbonyl groups to an arylene group is preferable.

Specific examples of the suitable cation represented by General Formula (ca-1) include cations each represented by Chemical Formulae (ca-1-1) to (ca-1-72) shown below.

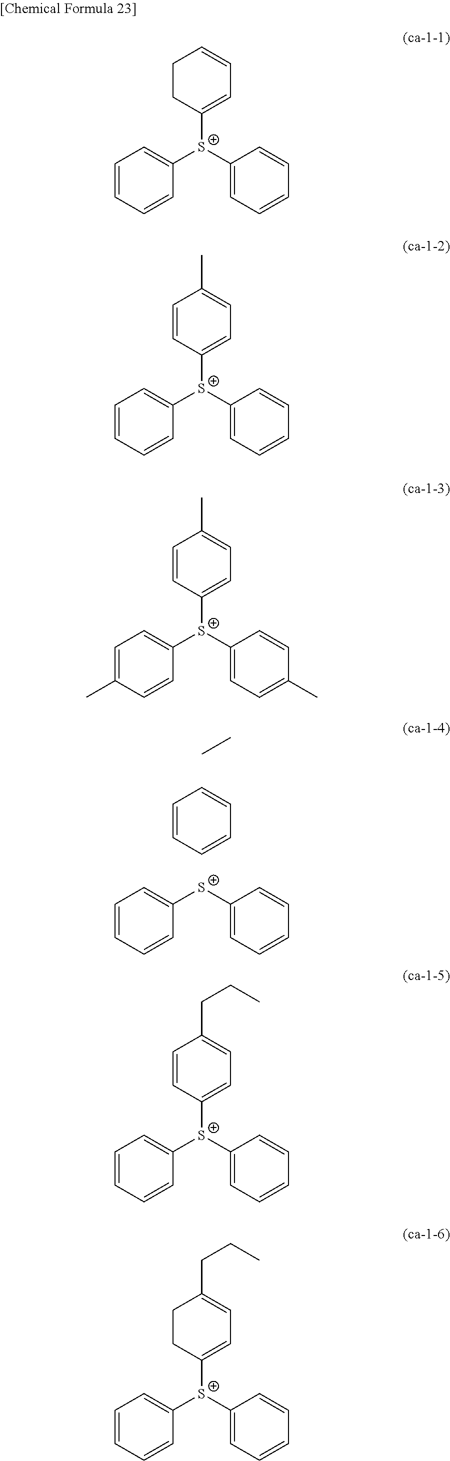

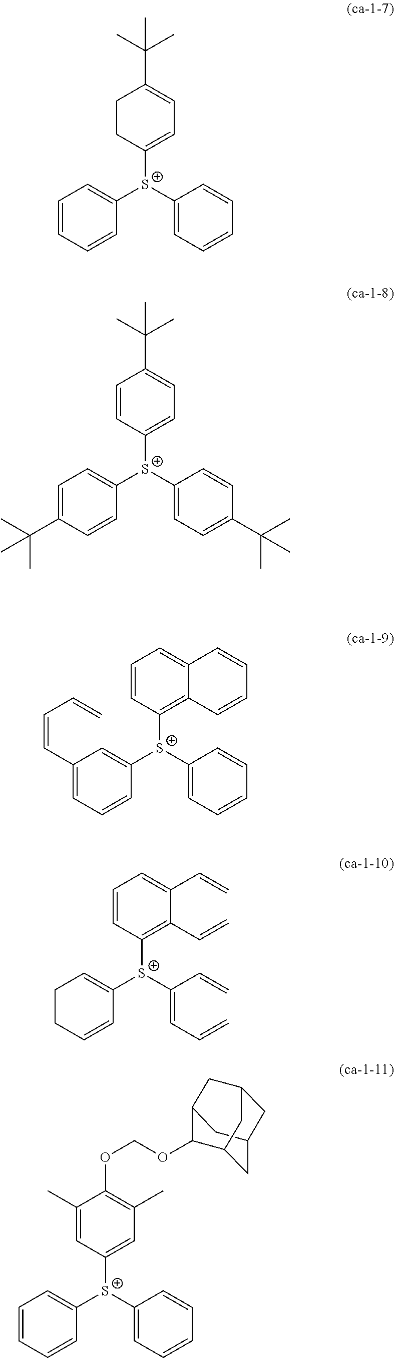

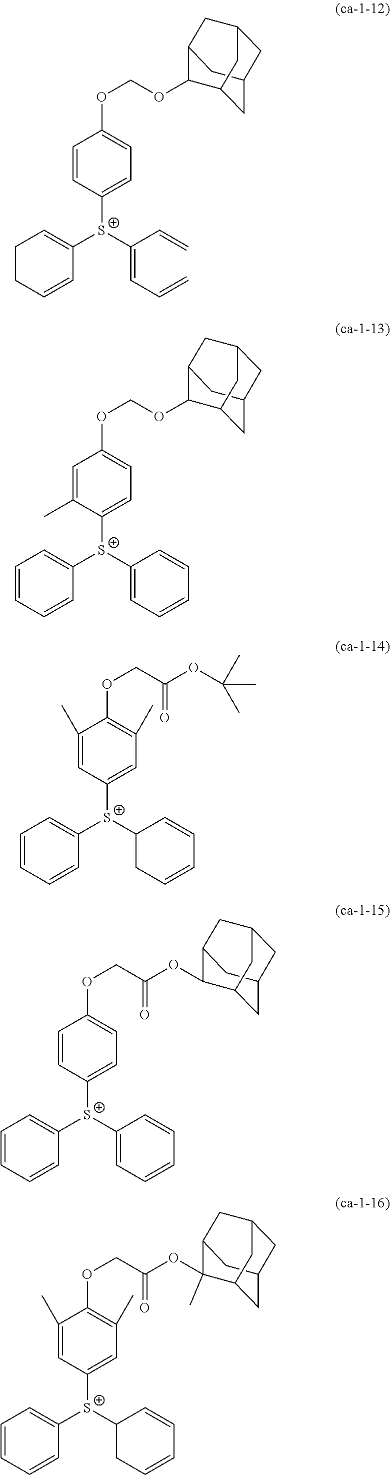

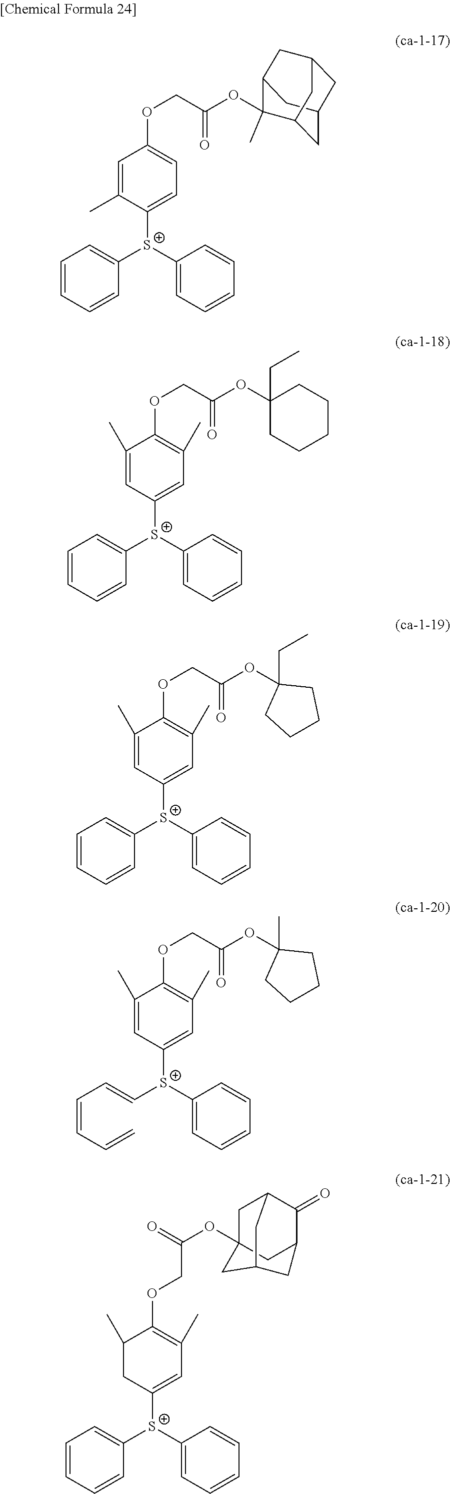

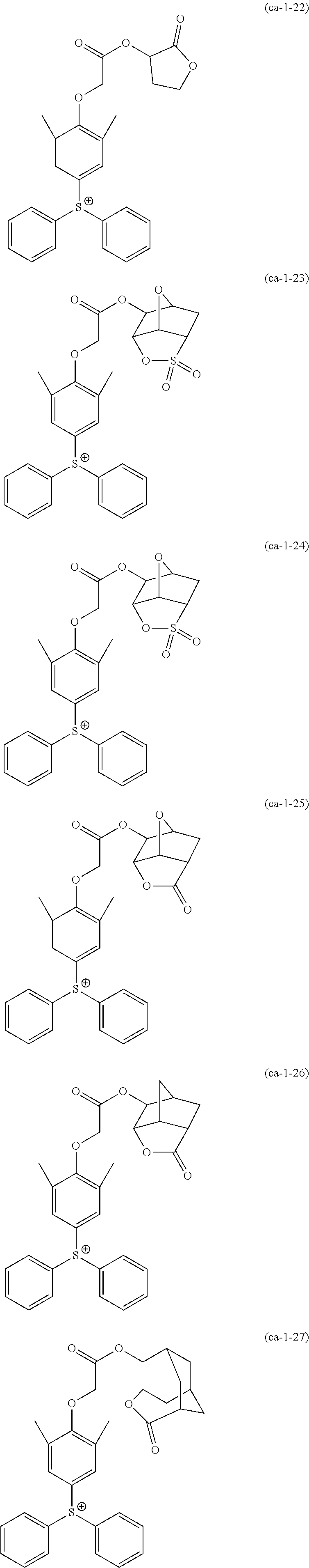

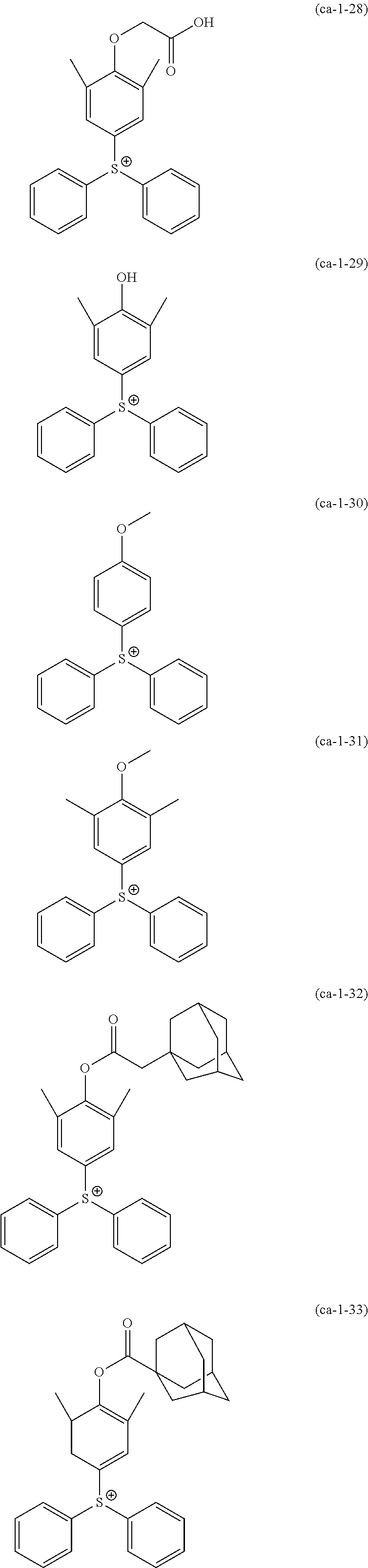

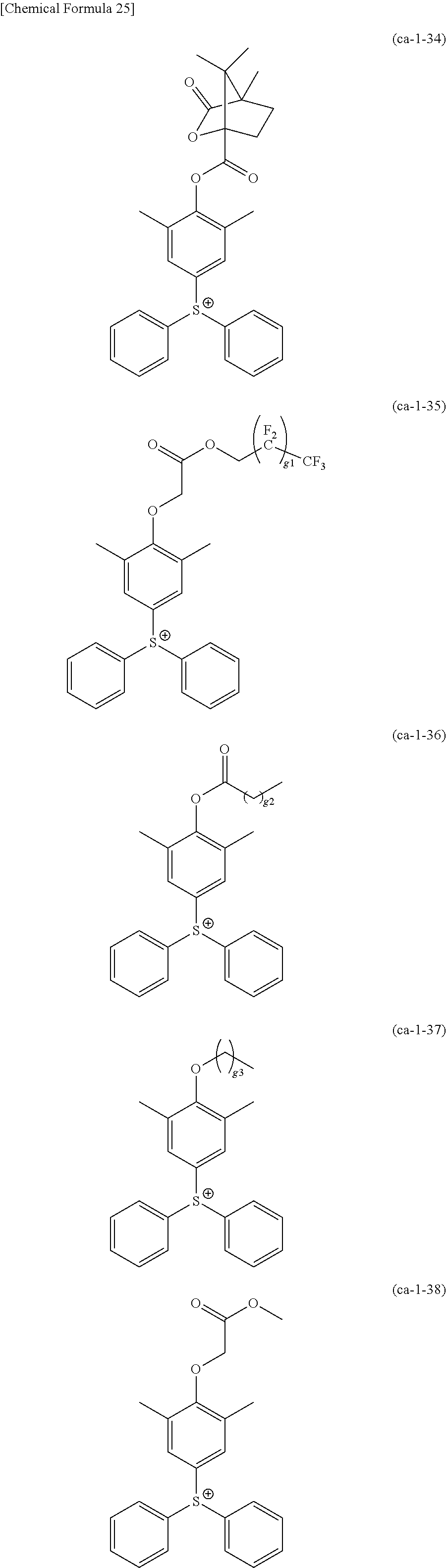

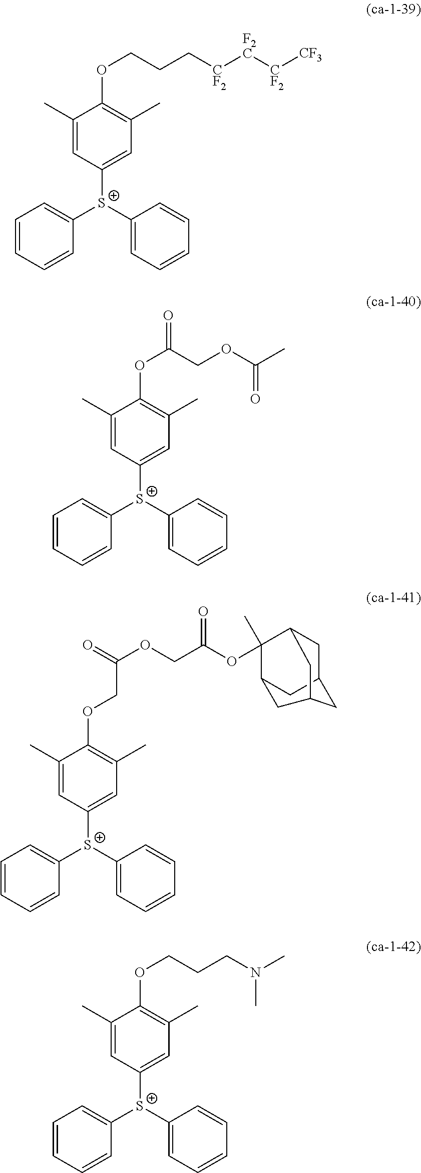

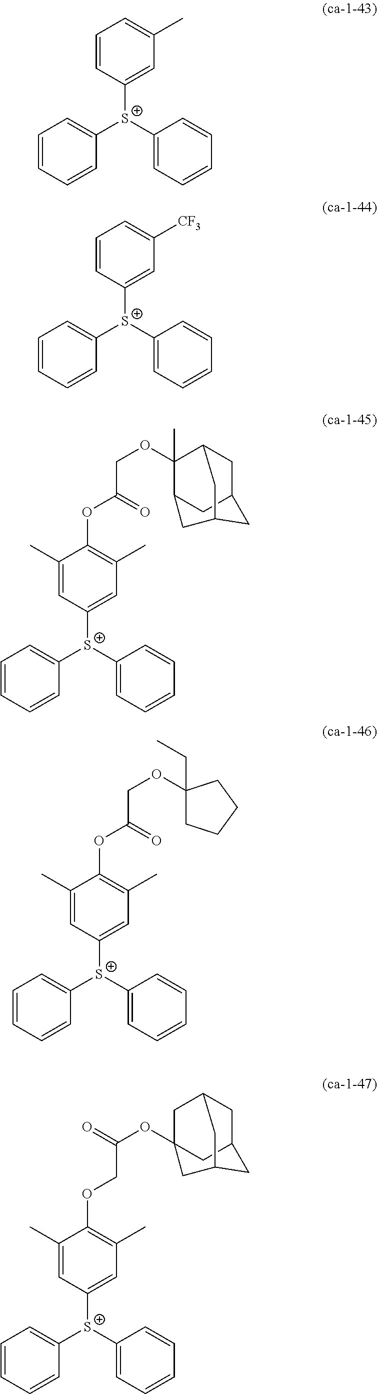

[In the formulae, g1, g2, and g3 represent a repeating number, g1 represents an integer of 1 to 5, g2 represents an integer of 0 to 20, and g3 represents an integer of 0 to 20.]

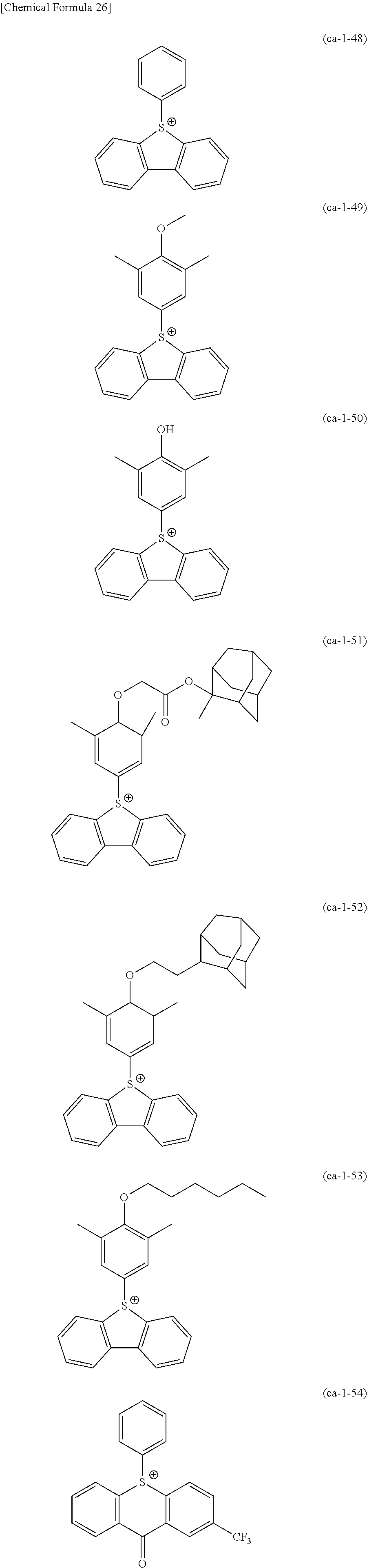

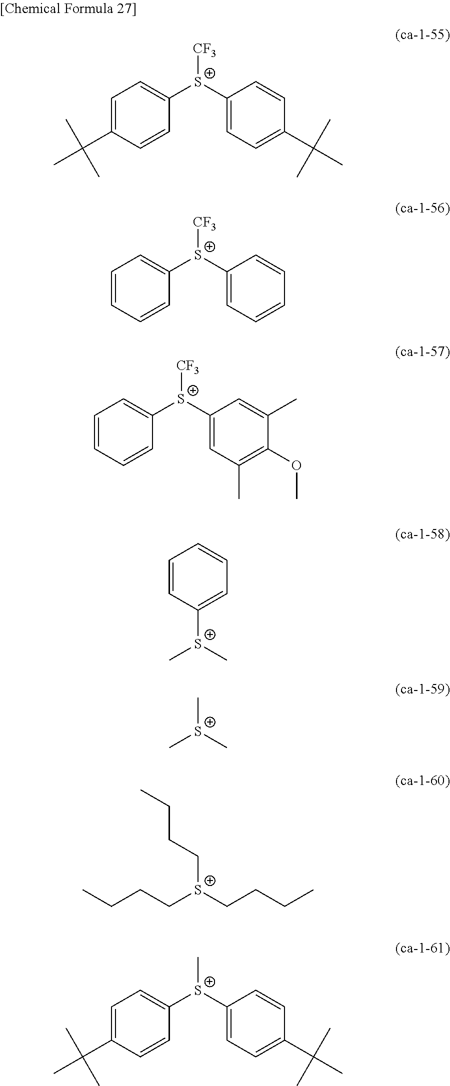

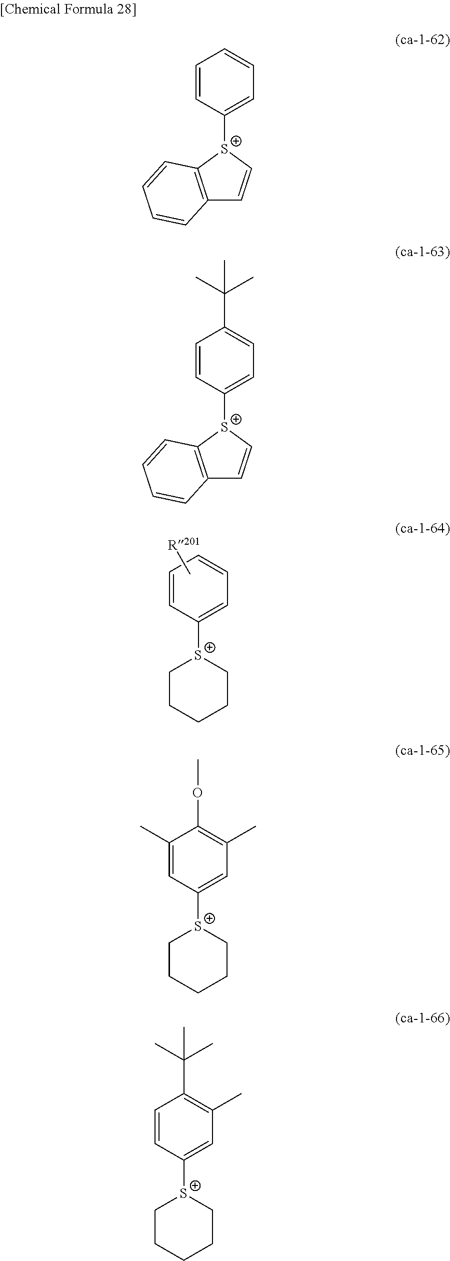

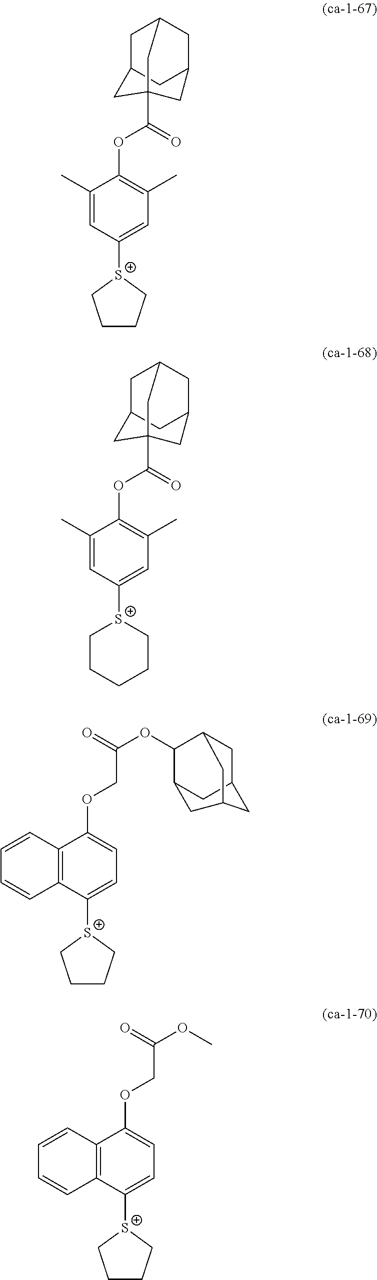 [SMILES: Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3 ; COC(=O)COc1ccc([S+]2CCCC2)c2ccccc12]

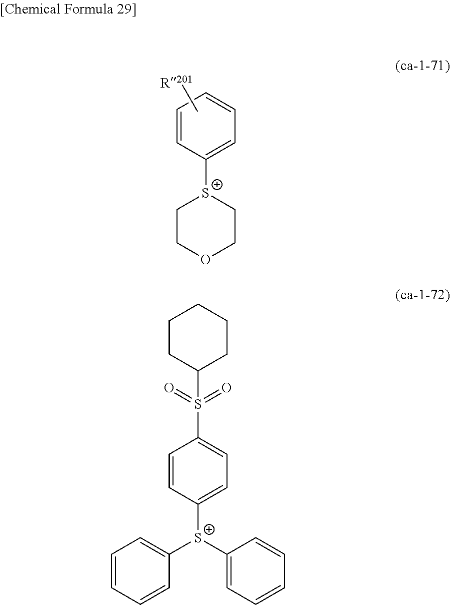

[In the formulae, R″²⁰¹ represents a hydrogen atom or a substituent, and examples of the substituent include the same groups as those for the substituents which may be contained in R²⁰¹ to R²⁰⁷ and R²¹⁰ to R²¹².

Specific examples of suitable cations represented by Formula (ca-2) include a diphenyliodonium cation and a bis(4-tert-butylphenyl)iodonium cation.

Specific examples of suitable cations represented by Formula (ca-3) include cations each represented by Formulae (ca-3-1) to (ca-3-6).

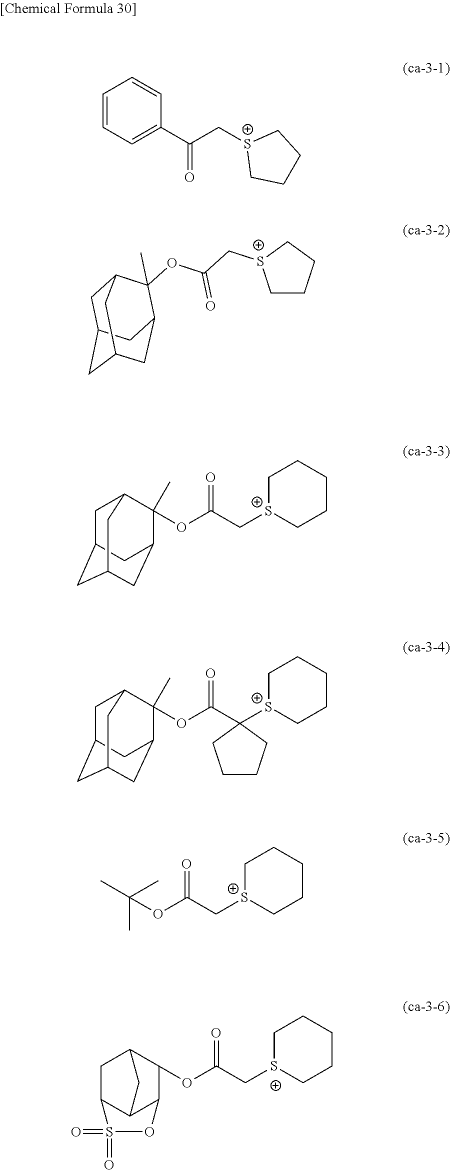 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

Specific examples of suitable cations represented by Formula (ca-4) include cations each represented by Formulae (ca-4-1) and (ca-4-2).

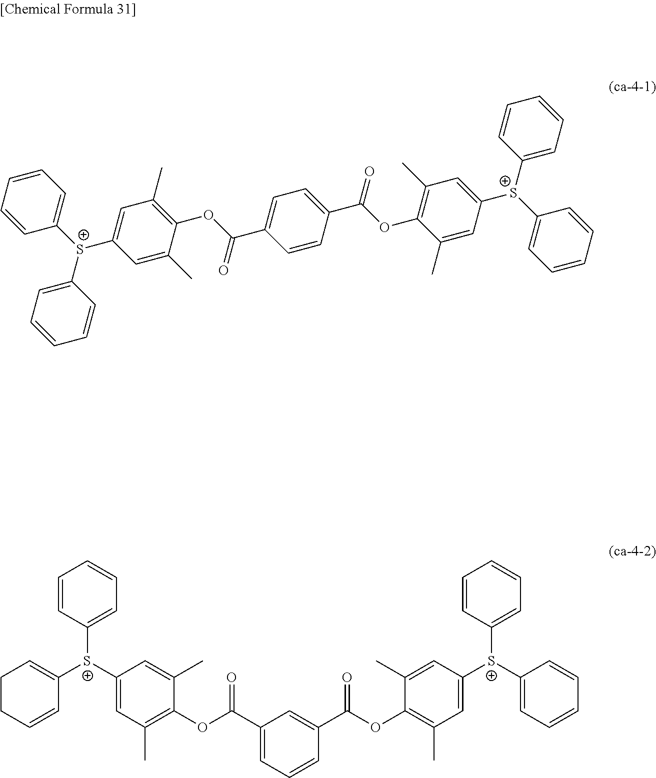 [SMILES: Cc1cc([S+](c2ccccc2)c2ccccc2)cc(C)c1OC(=O)c1ccc(C(=O)Oc2c(C)cc([S+](c3ccccc3)c3ccccc3)cc2C)cc1 ; Cc1cc([S+](C2=CCCC=C2)c2ccccc2)cc(C)c1OC(=O)c1cccc(C(=O)Oc2c(C)cc([S+](c3ccccc3)c3ccccc3)cc2C)c1]

Specific examples of suitable cations represented by Formula (ca-5) include cations each represented by General Formulae (ca-5-1) and (ca-5-3).

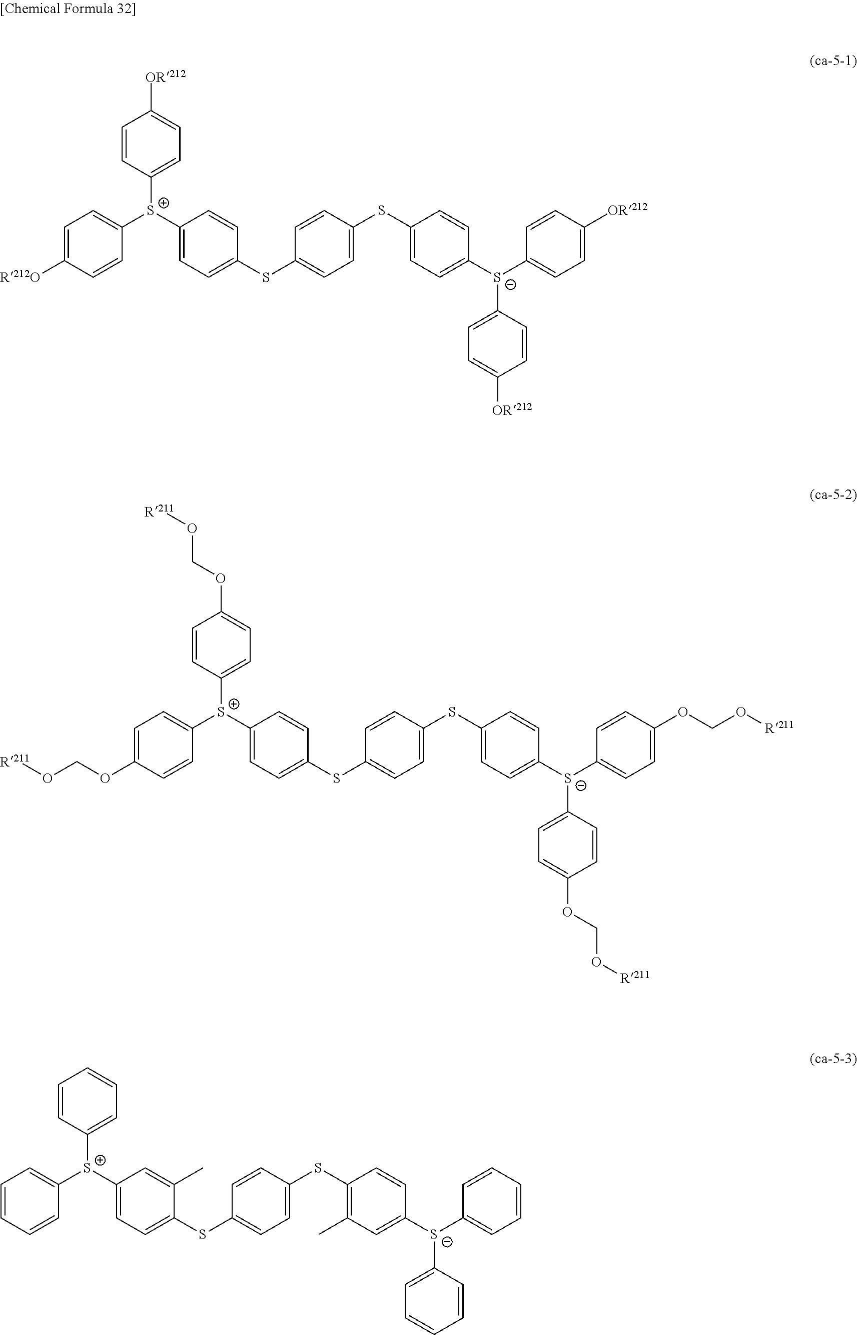

Among the above, the cation moiety ((M^{m+}))_{1/m}) is preferably a sulfonium cation and more preferably a cation represented by General Formula (ca-1).

### [Step (I)]

In the step (I) according to the present embodiment, a carboxylic acid (compound (d0-1)) represented by General Formula (d0-1) and having an acid dissociation constant (pKa) of 0.50 or more is reacted with at least one compound (compound (X0)) selected from the group consisting of a nitrogen-containing base compound and an onium compound to obtain an intermediate (compound (d0-p)) represented by General Formula (d0-p).

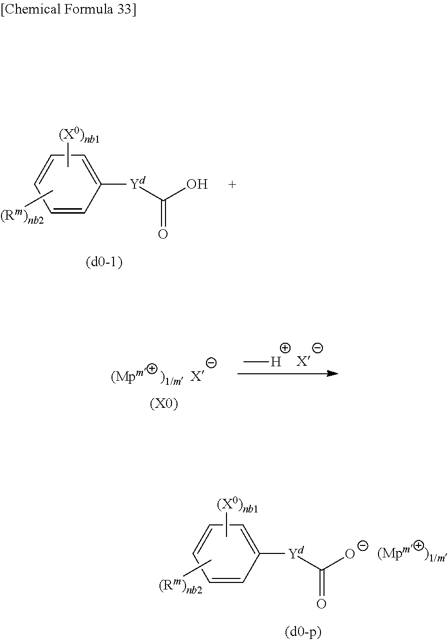

[X⁰, R^{m}, nb1, nb2, and Y^{d} in General Formula (d0-1) are the same as X⁰, R^{m}, nb1, nb2, and Y^{d} in General Formula (d0-1) described above.

Mp^{m′+}, m′, and X′⁻ in General Formula (X0) are the same as Mp^{m′+}, m′, and X′⁻ in General Formula (X0) described above.

X⁰, R^{m}, nb1, nb2, and Y^{d}, and Mp^{m′+} and m′ in General Formula (d0-p) are each the same as X⁰, R^{m}, nb1, nb2, and Y^{d} in General Formula (d0-1) described above and Mp^{m′+} and m′ in General Formula (X0) described above.]

In the step (I), the reaction General the compound (d0-1) and the compound (X0) is performed, for example, in water.

Regarding the mixing ratio of the compound (d0-1) and the compound (X0), for example, the compound (X0) is preferably 0.9 to 1.0 mole with respect to 1 mole of the compound (d0-1).

The reaction time of the step (I) is, for example, preferably 5 minutes or more and 24 hours or less, more preferably 10 minutes or more and 120 minutes or less, and still more preferably 10 minutes or more and 60 minutes or less.

The reaction temperature in the step (I) is preferably 0° C. or more and 50° C. or less, and more preferably 10° C. or more and 30° C. or less.

After the reaction between the compound (d0-1) and the compound (X0) is completed, the compound in the reaction solution may be isolated and purified. A known method in the related art can be used for isolation and purification, and for example, concentration, solvent extraction (liquid-liquid extraction), distillation, crystallization, recrystallization, or chromatography can be appropriately combined and used.

According to the present embodiment, in a case where solvent extraction (liquid-liquid extraction) is used, the recovery rate of the intermediate (compound (d0-p)) in the water phase can be increased after adding an organic solvent to the reaction solution and mixing the reaction solution. Examples of the organic solvent that can be used in such case include ketone-based solvents such as cyclohexanone, methyl ethyl ketone, diethyl ketone, and methyl isobutyl ketone.

Hereinafter, specific examples of the intermediate (compound (d0-p)) obtained in the step (I) are shown.

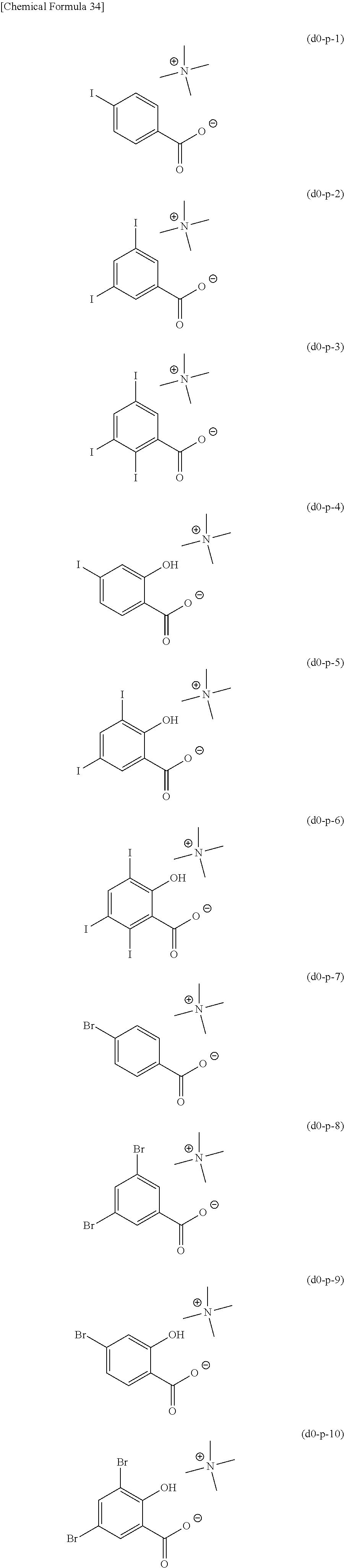

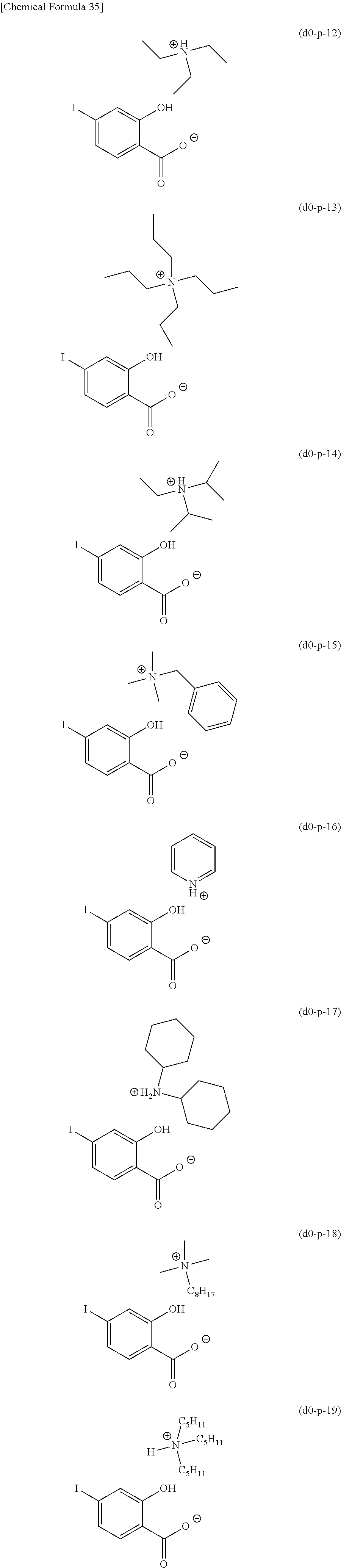

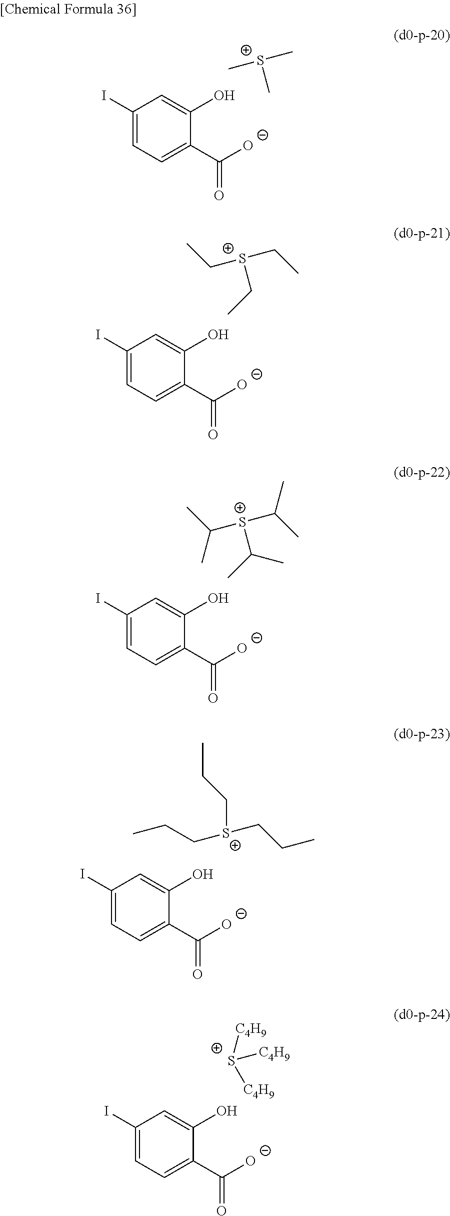

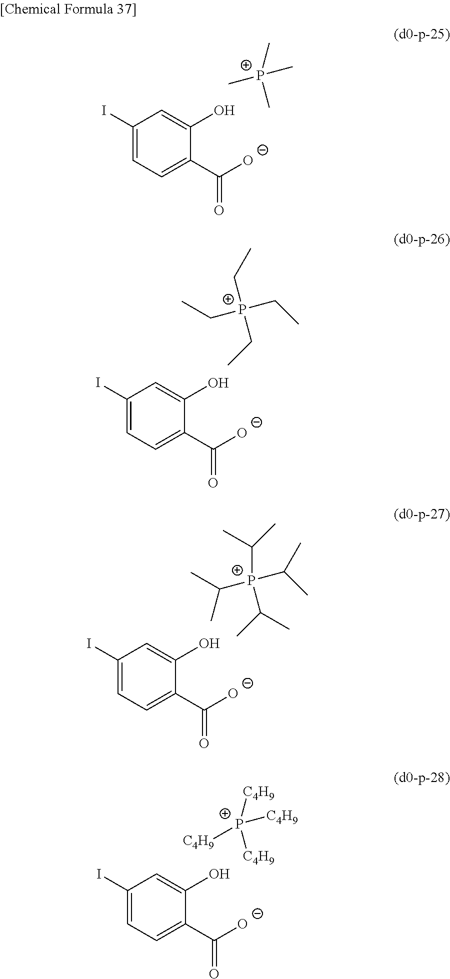

### [Step (II)]

In the step (II) according to the present embodiment, the intermediate (compound (d0-p)) obtained in the step (I) is subjected to an ion exchange reaction with the compound (compound (c0)) represented by General Formula (c0) to obtain a compound (compound (d0)) represented by General Formula (d0), which is the final target product.

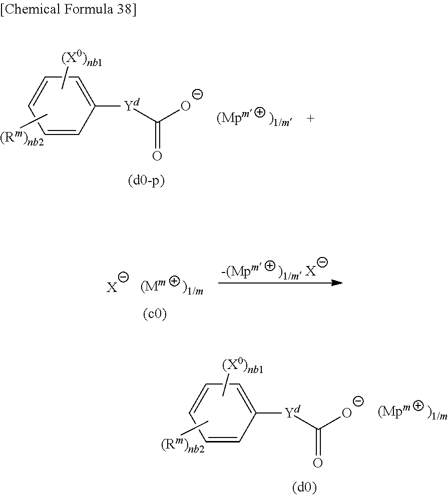

[X⁰, R^{m}, nb1, nb2, and Y^{d}, and Mp^{m′+} and m′ in General Formula (d0-p) are each the same as X⁰, R^{m}, nb1, nb2, and Y^{d} in General Formula (d0-1) described above and Mp^{m′+} and m′ in General Formula (X0) described above.

X⁻, M^{m+}, and m′ in General Formula (c0) are the same as X⁻, M^{m+}, and m in General Formula (c0) described above.

X⁰, R^{m}, nb1, nb2, and Y^{d}, and M^{m+} and m in General Formula (d0) are each the same as X⁰, R^{m}, nb1, nb2, and Y^{d} in General Formula (d0-1) described above and M^{m+} and m in General Formula (c0) described above.]

In the step (II), the ion exchange reaction between the compound (d0-p) and the compound (c0) is performed, for example, in a mixed solvent of an organic solvent and water.

Examples of the organic solvent include a ketone-based solvent such as cyclohexanone, methyl ethyl ketone, diethyl ketone, or methyl isobutyl ketone, an ether-based solvent such as diethyl ether, t-butyl methyl ether, or diisopropyl ether, a halogen-based solvent such as tetrahydrofuran, 1,3-dioxolane, dichloromethane, or 1,2-dichloroethane, an ester-based solvent such as ethyl acetate or propylene glycol monomethyl ether acetate, propionitrile, and a mixed solvent thereof.

Regarding the mixing ratio of the compound (d0-p) and the compound (c0), for example, the compound (c0) is preferably 0.9 to 1.0 mole with respect to 1 mole of the compound (d0-p).

The reaction time of the step (II) is, for example, preferably 5 minutes or more and 24 hours or less, more preferably 10 minutes or more and 120 minutes or less, and still more preferably 10 minutes or more and 60 minutes or less.

The reaction temperature in the step (II) is preferably 0° C. or more and 50° C. or less, and more preferably 10° C. or more and 30° C. or less.

After the ion exchange reaction between the compound (d0-p) and the compound (c0) is completed, the compound in the reaction solution may be isolated and purified. A known method in the related art can be used for isolation and purification, and for example, concentration, solvent extraction (liquid-liquid extraction), distillation, crystallization, recrystallization, or chromatography can be appropriately combined and used.

According to the present embodiment, in a case where solvent extraction (liquid-liquid extraction) is used, the recovery rate of the final target product (compound (d0)) in the organic phase can be increased. In addition, even in a case where washing with water is performed after the recovery from the organic phase, a high yield is maintained.

The structure of the final target product (compound (d0)) obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), an elemental analysis method, and an X-ray crystal diffraction method.

As the raw material used in each step, a commercially available raw material may be used, or a synthesized raw material may be used.

Hereinafter, specific examples of the final target product (compound (d0)) obtained in the step (II) are shown.

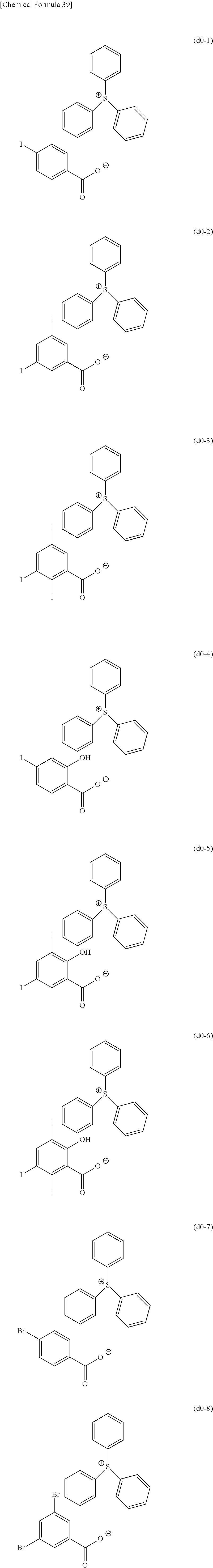

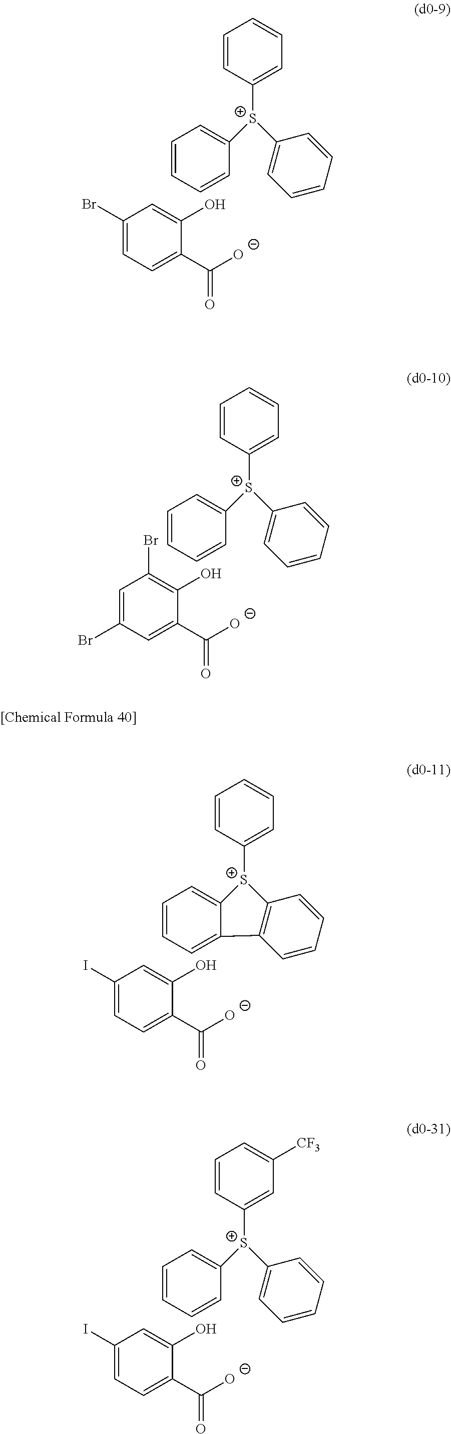

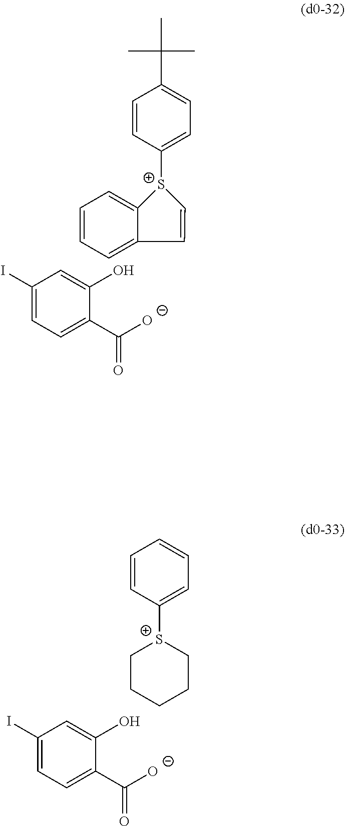 [SMILES: CC(C)(C)c1ccc(-[s+]2ccc3ccccc32)cc1 ; O=C([O-])c1ccc(I)cc1O ; c1ccc([S+]2CCCCC2)cc1 ; O=C([O-])c1ccc(I)cc1O]

The method for producing an acid generator according to the present embodiment described above includes a step (I) of reacting a carboxylic acid having a benzene ring in which a bromine atom or an iodine atom is bonded as a substituent and having an acid dissociation constant (pKa) of 0.50 or more with at least one selected from the group consisting of a nitrogen-containing base compound and an onium compound, which have a cation moiety with an octanol/water partition coefficient (logP_{OW}) of 4.8 or less to obtain an intermediate, and a step (II) of subjecting the intermediate obtained in the step (I) to an ion exchange reaction with an onium salt to obtain an acid generator which is a final target product.

In such production method, in the step (I), an intermediate having a cation moiety with a logP_{OW} of 4.8 or less and having increased water solubility is obtained. Therefore, a decrease in the recovery rate of the intermediate due to washing with an organic solvent or the like is suppressed. In addition, in the step (II), a lipophilic final target product having a benzene ring in which a bromine atom or an iodine atom is bonded as a substituent, the benzene ring being derived from the specific carboxylic acid used in the step (I), and having a pKa of the conjugate acid of 0.50 or more is obtained. Therefore, a decrease in the recovery rate of the final target product due to washing with water or the like is suppressed. In a case where the step (I) and the step (II) are combined, in the method for producing an acid generator according to the present embodiment, the acid generator for a resist composition can be produced with a higher yield.

The acid generator produced by the production method according to the present embodiment has a pKa of the acid generated upon exposure to light of 0.50 or more, and can be used as an acid diffusion control agent (photodecomposable base) in the resist composition in combination with an acid generator that generates an acid having a relatively low pKa.

In addition, the anion moiety of the acid generator has an iodine atom or a bromine atom that exhibits high absorbability with respect to EUV or EB. As a result, in the lithography using EUV or EB as a light source for exposure, an improvement in sensitivity is expected. Furthermore, in a case where the anion moiety has an iodine atom or a bromine atom, the hydrophobicity of the resist film is enhanced, and it is expected that the solubility in a developing solution can be appropriately adjusted. Therefore, the lithography characteristics can be further improved.

In the above-described embodiment, the production method having the step (I) and the step (II) has been described, but the present invention is not limited thereto, and may further have other steps as necessary in addition to the step (I) and the step (II).

### EXAMPLES

Hereinafter, the present invention will be described in more detail with reference to examples, but the present invention is not limited to these examples.

In the present example, the compound represented by Chemical Formula (d0-1-1) is denoted as a “compound (d0-1-1)”. Compounds represented by other chemical formulae are also denoted in the same manner.

### <Raw Materials Used>

Hereinafter, a carboxylic acid used as raw materials, at least one (compounds (X)) selected from the group consisting of a nitrogen-containing base compound and an onium compound, and a compound (compound (c0)) for an ion exchange reaction are shown.

### Carboxylic Acid

The pKa value of each of the compounds (d0-1-1) to (d0-1-10) and the compounds (d1-1-1) to (d1-1-4) is shown.

The pKa here is a value calculated using the software “Chem3D ver 15.1.0.144” (trade name, manufactured by Hulinks Inc.).

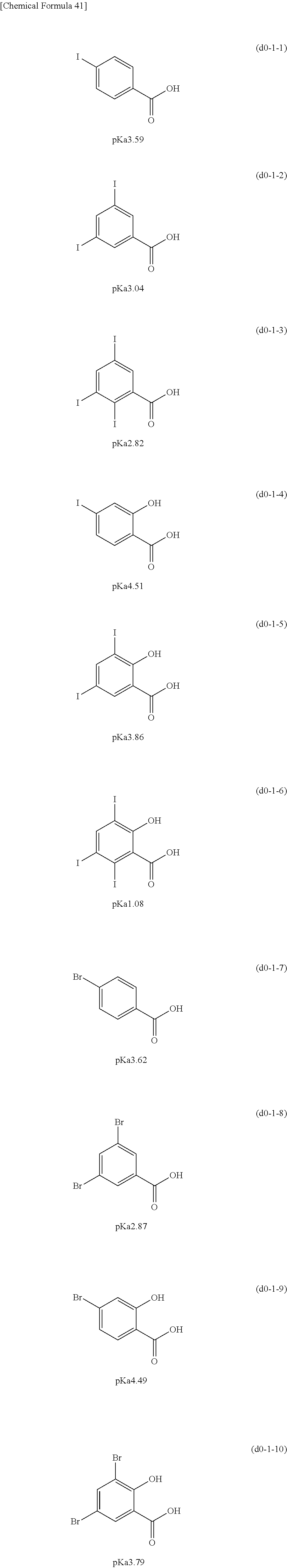

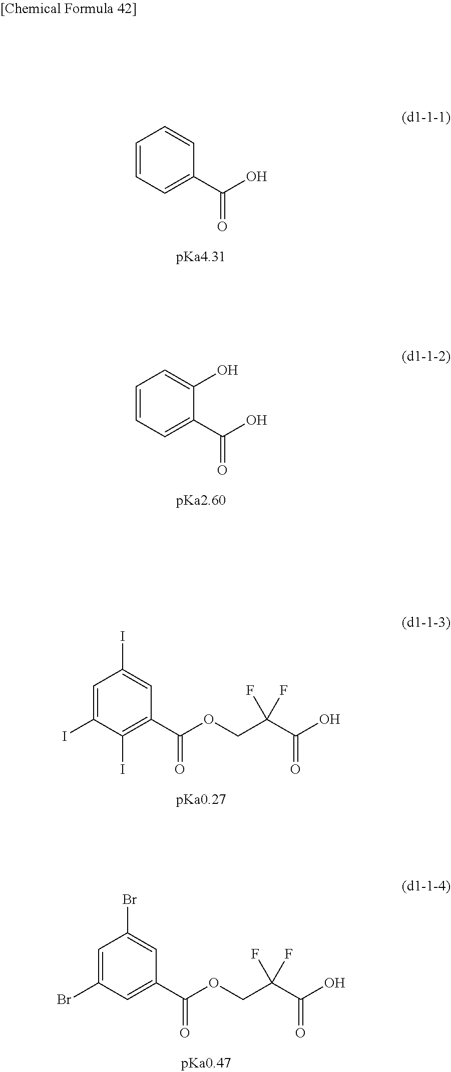 [SMILES: O=C(O)c1ccccc1 ; O=C(O)c1ccccc1O ; O=C(OCC(F)(F)C(=O)O)c1cc(I)cc(I)c1I ; O=C(OCC(F)(F)C(=O)O)c1cc(Br)cc(Br)c1]

At least one selected from the group consisting of nitrogen-containing base compound and onium compound (compound (X))

For each of the compounds (X0-1) to (X0-18) and the compounds (X1-1) to (X1-7), the logP_{OW} value of the cation moiety is shown.

The logP_{OW} here is a logP_{OW} value calculated by the CAChe Work System Pro Version 6.1.12.33.

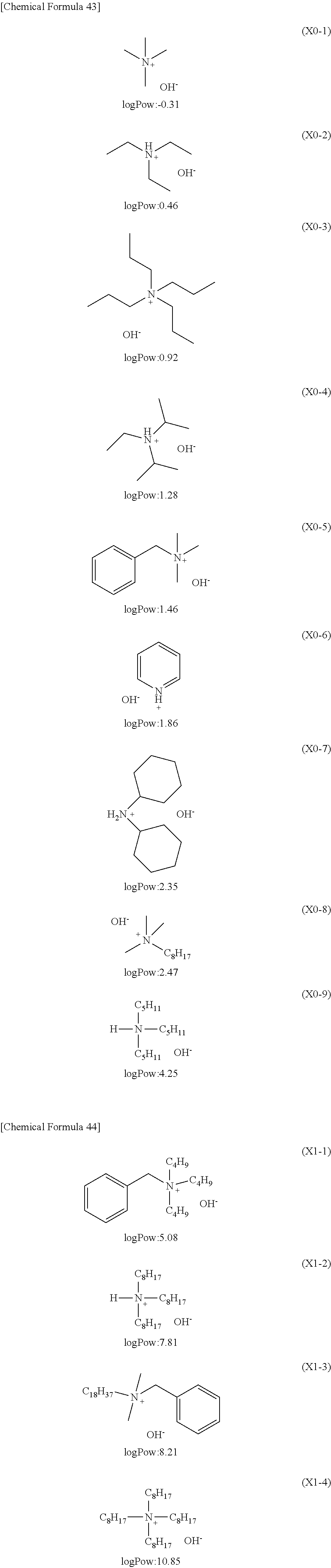

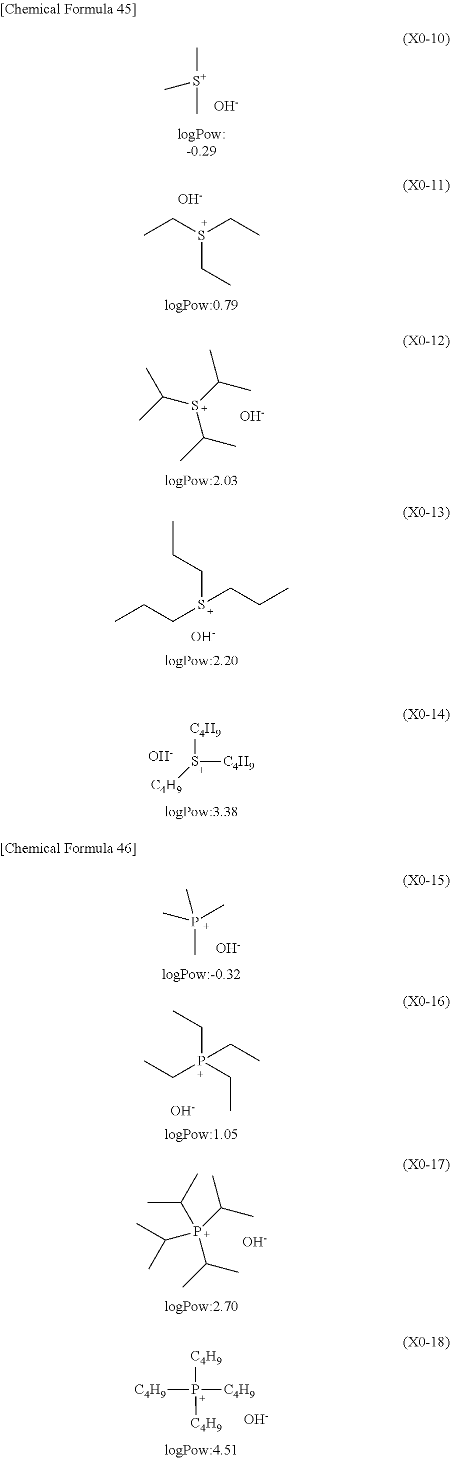

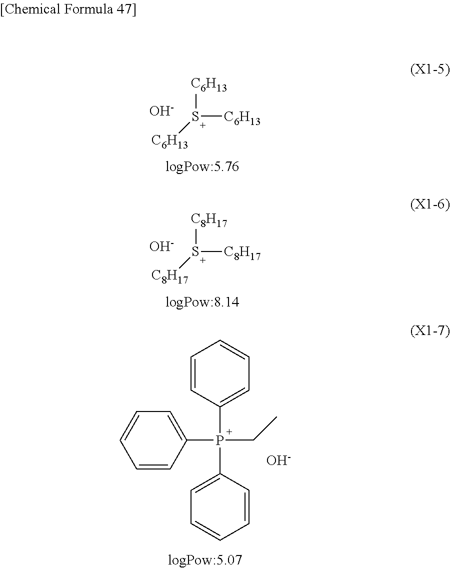

Compound for ion exchange reaction (compound (c0))

As the compound for the ion exchange reaction, the compounds (c0-1) to (c0-5) were used.

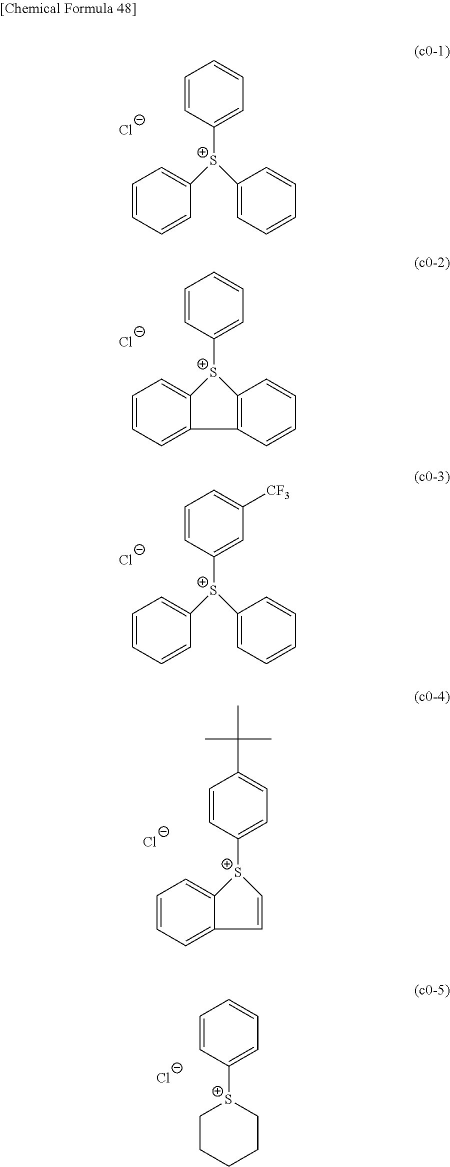

### <Method for Producing Acid Generator>

The production method of each example was performed by combining the raw materials as shown in Tables 1 to 4.

### Example 1

The production of the acid generator was performed using the compound (d0-1-1), the compound (X0-1), and the compound (c0-1) as raw materials.

### [Step (1): Step of Obtaining Intermediate]

15.90 g of 4-iodobenzoic acid (compound (d0-1-1)), 5.55 g of tetramethylammonium hydroxide (compound (X0-1)), and 50 g of water were put into a 200 mL beaker, and stirred at room temperature for 30 minutes to obtain a solution. 64 g of methyl isobutyl ketone was added to the solution and stirred at room temperature for 30 minutes, and then the solution was separated into layers and the water phase was collected to obtain an aqueous solution containing 18.58 g of an ammonium salt (compound (d0-p-1)) as an intermediate.

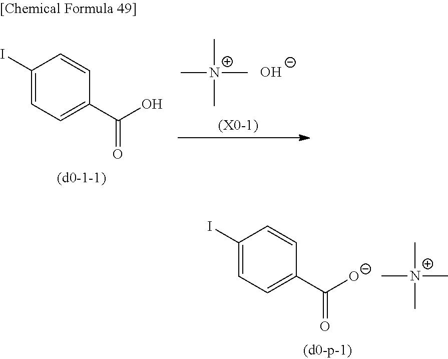

### [Step (II): Step of Obtaining Final Target Product]

16.44 g of the chloride salt (compound (c0-1)), 150 g of water, and 150 g of methyl isobutyl ketone were added to the aqueous solution of the ammonium salt (compound (d0-p-1)) obtained in the step (I), and stirred at room temperature for 30 minutes, and then the mixture was separated into layers, and the organic layer was collected. The obtained organic layer was washed with water and concentrated under reduced pressure to obtain 23.86 g of a sulfonium salt (compound (d0-1)) which was a final target product.

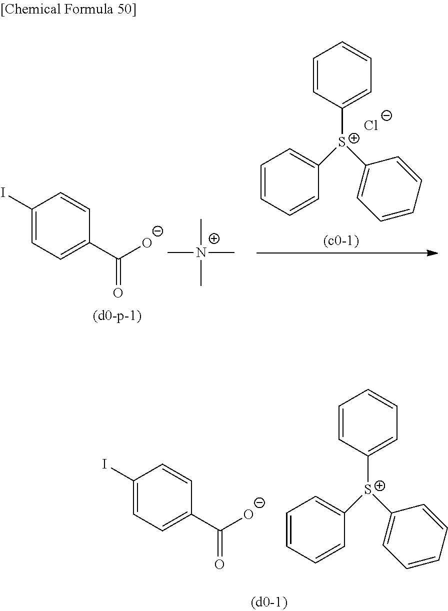

### Examples 2 to 33 and Comparative Examples 1 to 11

The operations of the step (I) and the step (II) in Example 1 were performed in the same manner except that the combination of the compounds (d0-1-1), the compound (X0-1), and the compound (c0-1) as the raw materials to be used was changed to each of the combinations of the raw materials shown in Tables 1 to 4, thereby obtaining the final target product via the intermediates.

Hereinafter, the intermediate obtained in the step (I) in each example is shown.

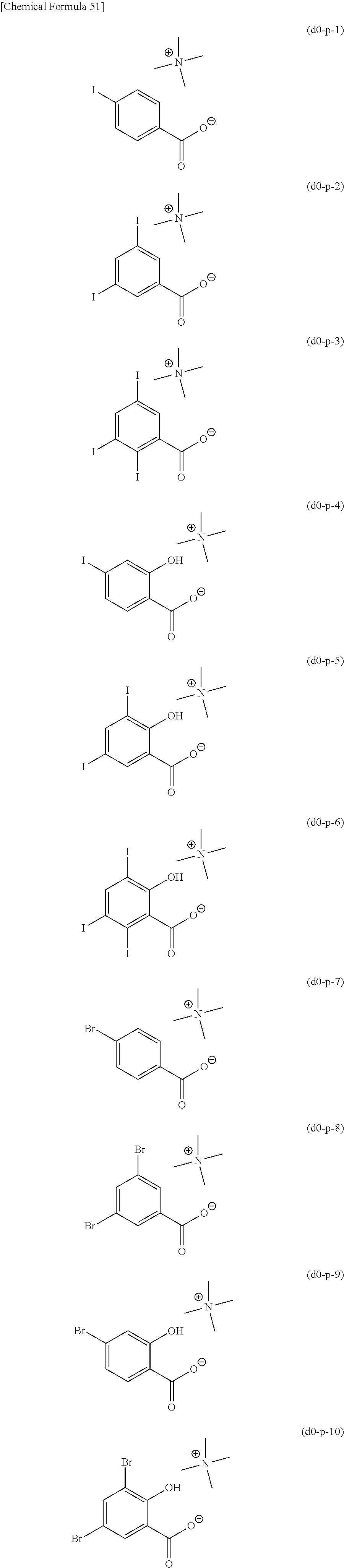

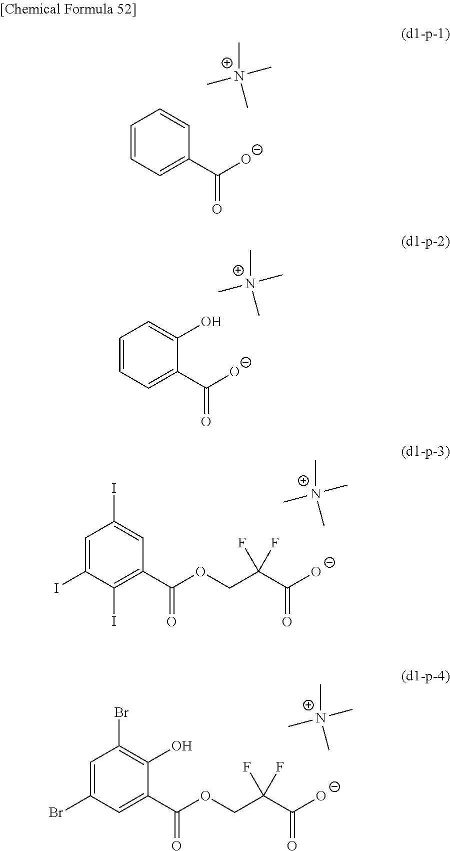

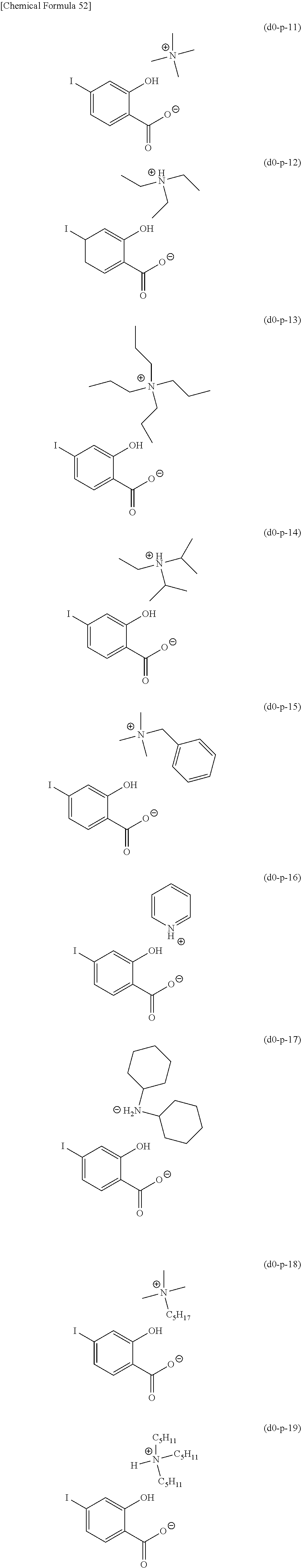

The compound (d0-p-11) has the same structure as the compound (d0-p-4).

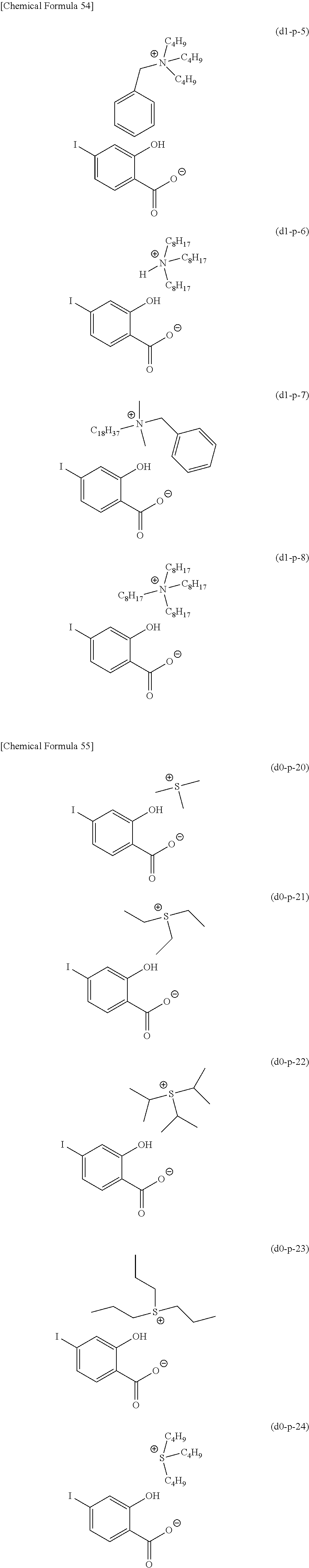

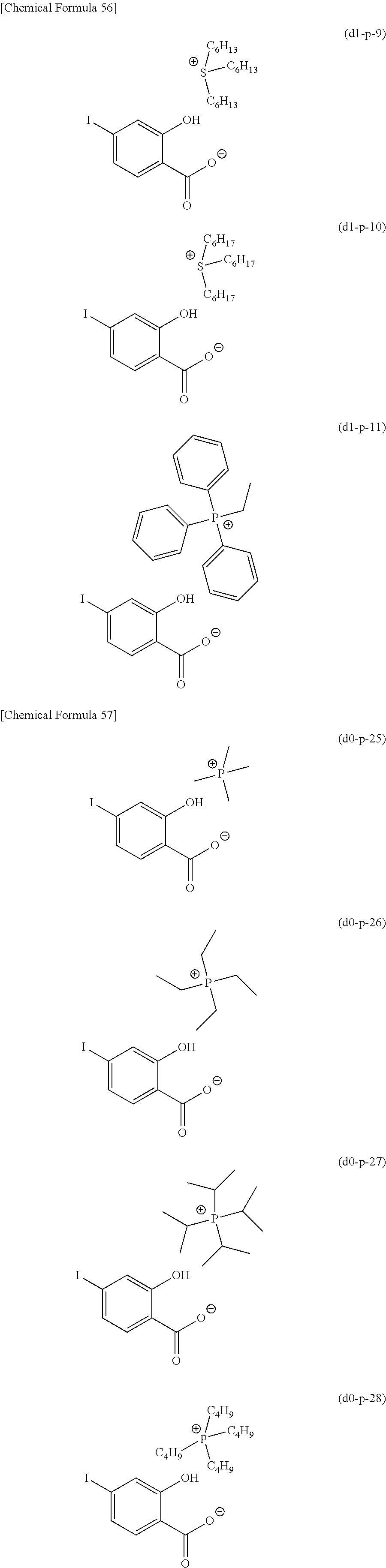

The compounds (d0-p-29) to (d0-p-33) which are intermediates obtained in the step (I) in Examples 29 to 33 are the same structure as the compound (d0-p-15) which is intermediate obtained in the step (I) in Example 15.

Hereinafter, the final target product obtained in the step (II) in each example is shown.

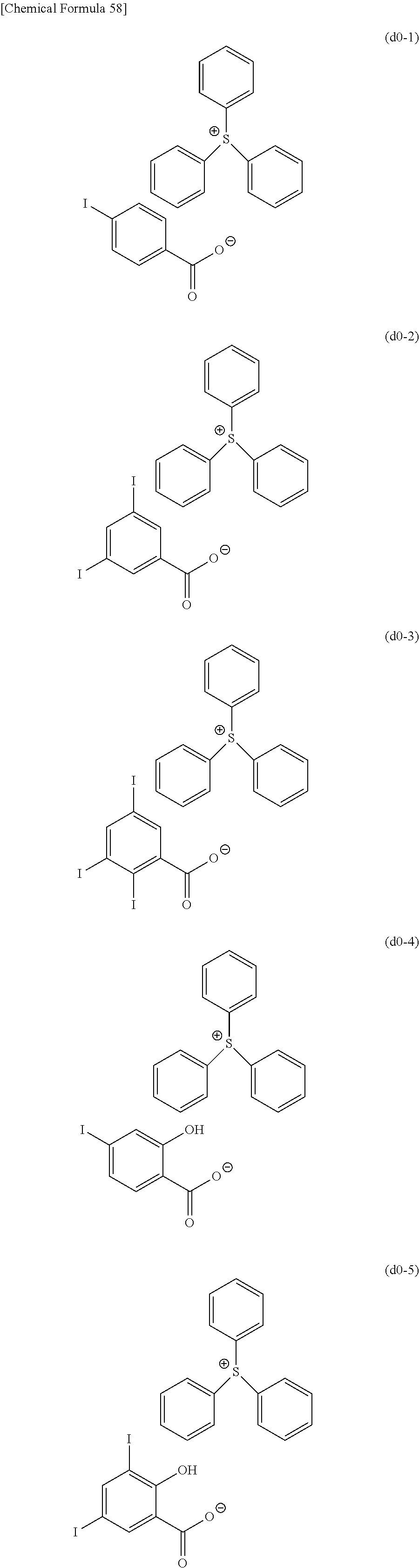

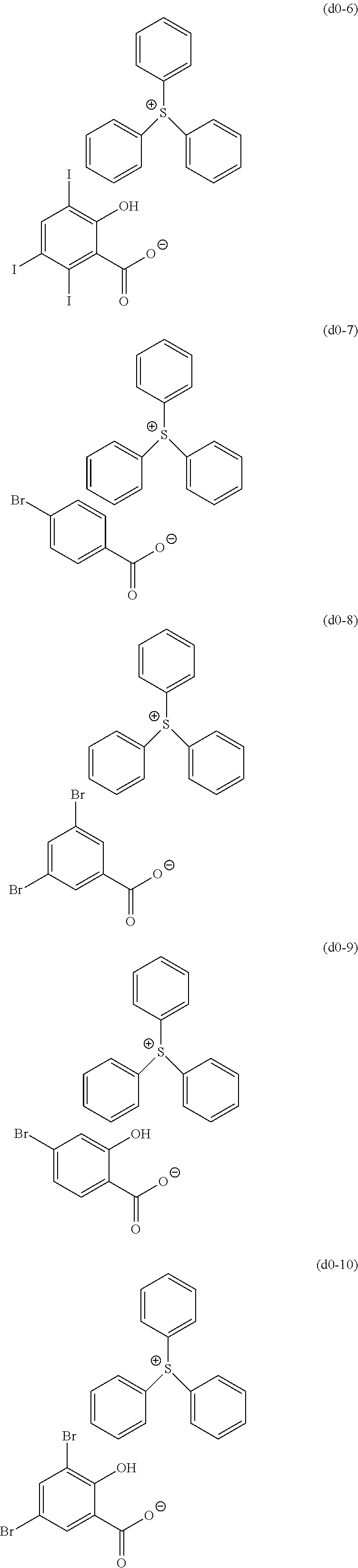

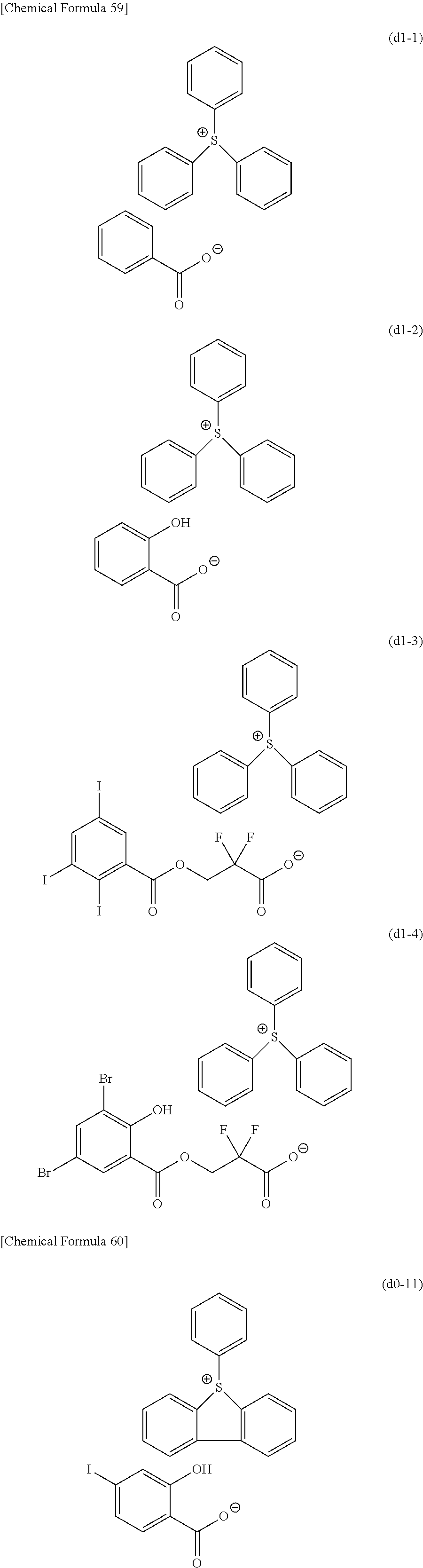

The compounds (d0-12) to (d0-28) which are final target products obtained in the step (II) in Examples 12 to 28 are the same structure as the compound (d0-11) which is final target product obtained in the step (II) in Example 11.

Similarly, the compounds (d1-5) to (d1-11) which are the final target products obtained in the step (II) in Comparative Examples 5 to 11 are the same structure as the compound (d0-11) which is the final target product obtained in the step (II) in Example 11.

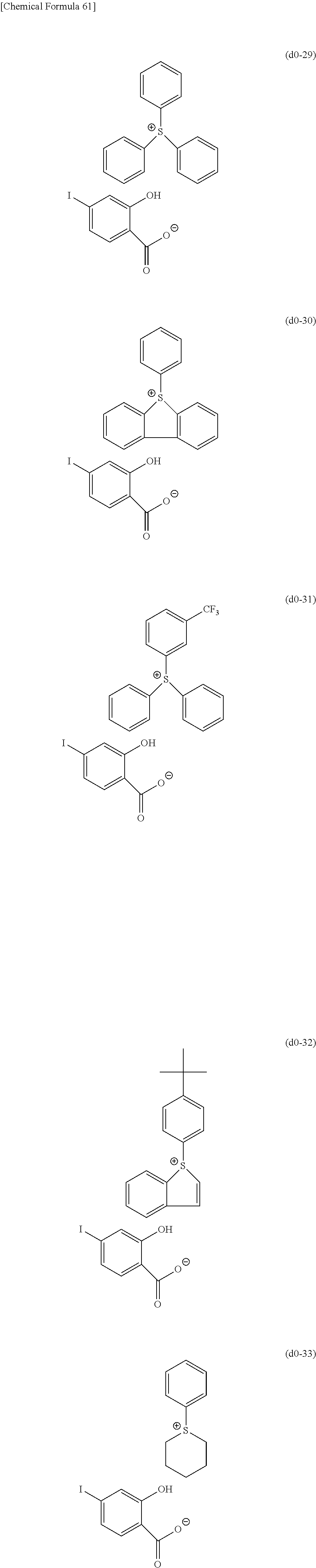

The compound (d0-29) has the same structure as the compound (d0-4). In addition, the compound (d0-30) has the same structure as the compound (d0-11).

### <Yield in Production Method of Each Example>

Tables 1 to 4 collectively show the raw materials used in the production method of each of the above-mentioned examples, the intermediates obtained in the production method of each of the above-mentioned examples, and the final target products finally obtained in the production method of each of the above-mentioned examples.

In addition, the yield in the production method of each example is shown in Tables 1 to 4. The yield here was calculated by multiplying the yield of the step (I) and the yield of the step (II). Specifically, the yield was obtained by the following calculation formula.

$$Yield ( \% ) = \frac{Amount of intermediate obtained in step ( I )}{\begin{matrix}( Number of moles of used compound ( X ) ) \times \\ ( Molecular weight of imtermediate )\end{matrix}} \times \frac{\begin{matrix}Amount of final target product \\ obtained in step ( II )\end{matrix}}{\begin{matrix}( Number of moles of used compound ( c 0 ) ) \times \\ ( Molecular weight of final target product )\end{matrix}} \times 100 \qquad [ Expression 1 ]$$

TABLE 1

| | Raw material | | | | | |
|---|---|---|---|---|---|---|
| | | At least one selected from | Compound | | | |
| | | group consisting of nitrogen- | for ion | | Final | |
| | Carboxylic | containing base compound | exchange | | target | Yield |
| | acid | and onium compound | reaction | Intermediate | product | % |
| Example 1 | (d0-1-1) | (X0-1) | (c0-1) | (d0-p-1) | (d0-1) | 91 |
| Example 2 | (d0-1-2) | (X0-1) | (c0-1) | (d0-p-2) | (d0-2) | 89 |
| Example 3 | (d0-1-3) | (X0-1) | (c0-1) | (d0-p-3) | (d0-3) | 85 |
| Example 4 | (d0-1-4) | (X0-1) | (c0-1) | (d0-p-4) | (d0-4) | 88 |
| Example 5 | (d0-1-5) | (X0-1) | (c0-1) | (d0-p-5) | (d0-5) | 86 |
| Example 6 | (d0-1-6) | (X0-1) | (c0-1) | (d0-p-6) | (d0-6) | 84 |
| Example 7 | (d0-1-7) | (X0-1) | (c0-1) | (d0-p-7) | (d0-7) | 90 |
| Example 8 | (d0-1-8) | (X0-1) | (c0-1) | (d0-p-8) | (d0-8) | 86 |
| Example 9 | (d0-1-9) | (X0-1) | (c0-1) | (d0-p-9) | (d0-9) | 87 |
| Example 10 | (d0-1-10) | (X0-1) | (c0-1) | (d0-p-10) | (d0-10) | 85 |
| Comparative | (d1-1-1) | (X0-1) | (c0-1) | (d1-p-1) | (d1-1) | 54 |
| Example 1 | | | | | | |
| Comparative | (d1-1-2) | (X0-1) | (c0-1) | (d1-p-2) | (d1-2) | 50 |
| Example 2 | | | | | | |
| Comparative | (d1-1-3) | (X0-1) | (c0-1) | (d1-p-3) | (d1-3) | 55 |
| Example 3 | | | | | | |
| Comparative | (d1-1-4) | (X0-1) | (c0-1) | (d1-p-4) | (d1-4) | 48 |
| Example 4 | | | | | | |

TABLE 2

| | Raw material | | | | | |
|---|---|---|---|---|---|---|
| | | At least one selected from | Compound | | | |
| | | group consisting of nitrogen- | for ion | | Final | |
| | Carboxylic | containing base compound | exchange | | target | Yield |
| | acid | and onium compound | reaction | Intermediate | product | % |
| Example 11 | (d0-1-4) | (X0-1) | (c0-2) | (d0-p-11) | (d0-11) | 90 |
| Example 12 | (d0-1-4) | (X0-2) | (c0-2) | (d0-p-12) | (d0-12) | 82 |
| Example 13 | (d0-1-4) | (X0-3) | (c0-2) | (d0-p-13) | (d0-13) | 80 |
| Example 14 | (d0-1-4) | (X0-4) | (c0-2) | (d0-p-14) | (d0-14) | 79 |
| Example 15 | (d0-1-4) | (X0-5) | (c0-2) | (d0-p-15) | (d0-15) | 78 |
| Example 16 | (d0-1-4) | (X0-6) | (c0-2) | (d0-p-16) | (d0-16) | 75 |
| Example 17 | (d0-1-4) | (X0-7) | (c0-2) | (d0-p-17) | (d0-17) | 73 |
| Example 18 | (d0-1-4) | (X0-8) | (c0-2) | (d0-p-18) | (d0-18) | 71 |
| Example 19 | (d0-1-4) | (X0-9) | (c0-2) | (d0-p-19) | (d0-19) | 69 |
| Comparative | (d0-1-4) | (X1-1) | (c0-2) | (d1-p-5) | (d1-5) | 55 |
| Example 5 | | | | | | |
| Comparative | (d0-1-4) | (X1-2) | (c0-2) | (d1-p-6) | (d1-6) | 51 |
| Example 6 | | | | | | |
| Comparative | (d0-1-4) | (X1-3) | (c0-2) | (d1-p-7) | (d1-7) | 48 |
| Example 7 | | | | | | |
| Comparative | (d0-1-4) | (X1-4) | (c0-2) | (d1-p-8) | (d1-8) | 45 |
| Example 8 | | | | | | |

TABLE 3

| | Raw material | | | | | |
|---|---|---|---|---|---|---|
| | | At least one selected from | Compound | | | |
| | | group consisting of nitrogen- | for ion | | Final | |
| | Carboxylic | containing base compound | exchange | | target | Yield |
| | acid | and onium compound | reaction | Intermediate | product | % |
| Example 20 | (d0-1-4) | (X0-10) | (c0-2) | (d0-p-20) | (d0-20) | 83 |
| Example 21 | (d0-1-4) | (X0-11) | (c0-2) | (d0-p-21) | (d0-21) | 81 |
| Example 22 | (d0-1-4) | (X0-12) | (c0-2) | (d0-p-22) | (d0-22) | 75 |
| Example 23 | (d0-1-4) | (X0-13) | (c0-2) | (d0-p-23) | (d0-23) | 73 |
| Example 24 | (d0-1-4) | (X0-14) | (c0-2) | (d0-p-24) | (d0-24) | 69 |
| Comparative | (d0-1-4) | (X1-5) | (c0-2) | (d1-p-9) | (d1-9) | 52 |
| Example 9 | | | | | | |
| Comparative | (d0-1-4) | (X1-6) | (c0-2) | (d1-p-10) | (d1-10) | 50 |
| Example 10 | | | | | | |
| Example 25 | (d0-1-4) | (X0-15) | (c0-2) | (d0-p-25) | (d0-25) | 91 |
| Example 26 | (d0-1-4) | (X0-16) | (c0-2) | (d0-p-26) | (d0-26) | 79 |
| Example 27 | (d0-1-4) | (X0-17) | (c0-2) | (d0-p-27) | (d0-27) | 70 |
| Example 28 | (d0-1-4) | (X0-18) | (c0-2) | (d0-p-28) | (d0-28) | 68 |
| Comparative | (d0-1-4) | (X1-7) | (c0-2) | (d1-p-11) | (d1-11) | 56 |
| Example 11 | | | | | | |

TABLE 4

| | Raw material | | | | | |
|---|---|---|---|---|---|---|
| | | At least one selected from | Compound | | | |
| | | group consisting of nitrogen- | for ion | | Final | |
| | Carboxylic | containing base compound | exchange | | target | Yield |
| | acid | and onium compound | reaction | Intermediate | product | % |
| Example 29 | (d0-1-4) | (X0-5) | (c0-1) | (d0-p-29) | (d0-29) | 77 |
| Example 30 | (d0-1-4) | (X0-5) | (c0-2) | (d0-p-30) | (d0-30) | 79 |
| Example 31 | (d0-1-4) | (X0-5) | (c0-3) | (d0-p-31) | (d0-31) | 78 |
| Example 32 | (d0-1-4) | (X0-5) | (c0-4) | (d0-p-32) | (d0-32) | 76 |
| Example 33 | (d0-1-4) | (X0-5) | (c0-5) | (d0-p-33) | (d0-33) | 77 |

From the results shown in Tables 1 to 4, it was confirmed that, according to the production method of the example to which the present invention was applied, the acid generator for a resist composition can be produced with a higher yield.

From the results of Examples 11 to 19 and Comparative Examples 5 to 8, Examples 20 to 24 and Comparative Examples 9 and 10, and Examples 25 to 28 and Comparative Example 11, it is found that the lower the octanol/water partition coefficient (logP_{OW}) of the cation moiety constituting the intermediate is, the higher the yield tends to be.

What is claimed is:

1. A method for producing an acid generator, the method comprising:
reacting a carboxylic acid represented by General Formula (d0-1) having an acid dissociation constant (pKa) of 0.50 or more with at least one selected from the group consisting of a nitrogen-containing base compound and an onium compound to obtain an intermediate represented by General Formula (d0-p); and
subjecting the intermediate obtained in the step (I) to an ion exchange reaction with a compound represented by General Formula (c0) to obtain a compound represented by General Formula (d0), 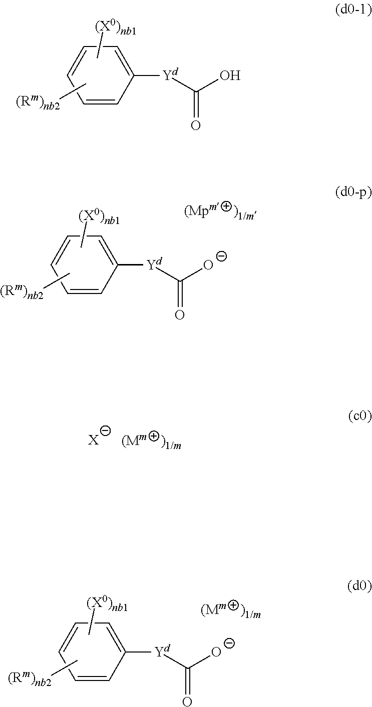
wherein X⁰ represents a bromine atom or an iodine atom, R^{m} represents a hydroxy group, an alkyl group, a fluorine atom, or a chlorine atom, nblI represents an integer in a range of 1 to 5 and nb2 represents an integer in a range of 0 to 4, where 1≤nb1+nb2≤5 is satisfied, Y^{d} represents a divalent linking group or a single bond, Mp^{m′+} represents an organic ammonium cation having an octanol/water partition coefficient (logP_{OW}) of 4.8 or less or an onium cation having a logP_{OW} of 4.8 or less, m′ represents an integer of 1 or more, X⁻ represents a counter anion, M^{m+} represents an onium cation, and m represents an integer of 1 or more.

2. The method for producing an acid generator according to claim 1, wherein the M^{m+} is a sulfonium cation.
